(19) United States
(12) Patent Application Publication
(10) Pub. No.: US 2010/0184049 A1
(43) Pub. Date: Jul. 22, 2010

(54) Glycoprotein Profiling of Bladder Cancer

(76) Inventors: Steve Goodison, Gainesville, FL (US); Charles Joel Rosser, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/597,693

(22) PCT Filed: Apr. 25, 2008
(86) PCT No.: PCT/US08/61599

§ 371 Date: Mar. 31, 2010

Related U.S. Application Data
(60) Provisional application No. 60/914,404, filed on Apr. 27, 2007.

(51) Int. Cl. G01N 33/574; C12Q 1/68; G01N 27/26

(52) U.S. Cl. 435/6; 436/501; 435/7.92; 204/450

(57) ABSTRACT

The present invention relates to a method for the diagnosis, prognosis, and monitoring of bladder cancer, such as early or late stage bladder cancer, by detecting in a urine sample from a subject at least one biomarker for bladder cancer identified herein, such as alpha-1B-glycoprotein, haptoglobin, serotransferrin, or alpha-1-antitrypsin. The biomarkers may be detected and, optionally, measured using an agent that detects or binds to the biomarker protein or an agent that detects or binds to encoding nucleic acids, such as antibodies specifically reactive with the biomarker protein or a portion thereof. The invention further relates to kits for carrying out the methods of the invention. The invention further relates to a device for the rapid detection of one or more bladder cancer biomarkers in urine and methods for rapidly measuring bladder cancer biomarkers in urine.

### CROSS-REFERENCE TO RELATED APPLICATION

The present application claims the benefit of U.S. Provisional Application Ser. No. 60/914,404, filed Apr. 27, 2007, which is hereby incorporated by reference herein in its entirety, including any figures, tables, nucleic acid sequences, amino acid sequences, and drawings.

### GOVERNMENT SUPPORT

The subject matter of this application has been supported by a research grant from the National Institutes of Health under grant number RO1 CA108597. Accordingly, the government has certain rights in this invention.

### BACKGROUND OF THE INVENTION

Cancer of the urinary bladder is among the five most common malignancies world-wide (Pisani, P. et al. Int J Cancer, 1999, 83(1):18-29). Transitional cell carcinomas (TCCs) are the most common urothelial tumors in Western countries and constitute approximately 95% of all cases (Aben, K. K. and Kiemeney, L. A. Eur Urol, 1999, 36(6):660-72). Early detection remains one of the most urgent issues in bladder cancer research. When detected early, the 5-year survival rate is approximately 94%; thus, timely intervention dramatically increases patient survival rate. Urothelial tumors can be classified into two groups based on histopathology and clinical behavior. At presentation, more than 80% of bladder tumors are non-muscle invasive papillary tumors (pTa or pT1). The remaining 20% of tumors that show muscle invasion at the time of diagnosis have a much less favorable prognosis. While radical surgery is required for invasive bladder tumors, superficial lesions are treated more conservatively by transurethral resection. However, more than 70% of patients with Ta/Tl lesions confined to the mucosa have recurrence during the first two years. If left untreated, these initially superficial lesions can progress to being muscle-invasive (Millan-Rodriguez, F. et al. J Urol, 2000, 164(3 Pt 1):680-4). The recurrence phenomenon of superficial bladder tumors makes bladder cancer one of the most prevalent cancers world-wide. Patients with superficial tumors are under continued surveillance by routine cystoscopy examinations of the bladder for early detection of new tumor developments. Once bladder tumors are identified and removed, patients will routinely get surveillance cystoscopy every 3 months for 2 years, then every 6 months for 2 years, then yearly thereafter. Consequently, the development of non-invasive urinalysis assays using reliable diagnostic markers would be of tremendous benefit to both patients and healthcare providers.

Voided urine cytology (VUC) remains the method of choice for the noninvasive detection of bladder cancer, with its main use being to recognize the presence of recurrence and early progression in stage and grade. VUC can be used to diagnose new malignancy, yet whilst it has a specificity of >93%, its sensitivity is only 25-40%, especially for low-grade and low-stage tumors (Cajulis, R. S. et al. Diagn Cytopathol, 1995, 13(3):214-23). Furthermore, results are not available rapidly, it is prone to inter-observer variation, and it is relatively expensive. Understandably, a good deal of research has focused on identifying potential urine tumor markers with higher sensitivity than urine cytology. Promising diagnostic protein markers are NMP-22, BTA, BLCA-4, and cell proliferation proteins. Unfortunately, these tests also suffer from high false-positive rates and thus there is no protein test available to date that can replace urine cytology (Hautmann, S. et al. Eur Urol, 2004, 46(4):466-71). Thus, detecting bladder cancer using diagnostic markers still remains a challenge.

Newly developed, high-throughput techniques, i.e., time-of-flight mass spectrometry, SDS/PAGE with matrix-assisted laser desorption/ionization ion-trap mass spectrometry, and liquid-phase 2-D separation techniques greatly facilitate the analysis of proteins in biological samples (Kreunin, P. et al. Proteomics, 2004, 4(9):2754-65). However, only a limited number of proteomic studies utilizing the newer technologies have been conducted in the analysis of urological cancers, largely because of the lack of defined methodologies that can reduce the complexity of the sample and rapidly and accurately identify specific proteins.

Recent improvements in technologies to detect, identify, and characterize proteins, particularly two-dimensional electrophoresis and mass spectrometry, enable the detailed and systematic isolation, identification and characterization of proteins in a given sample. Proteomics is regarded as a sister technology to genomics, however, although the pattern of gene activity may be abnormal in a tissue with a pathological lesion, there can be a poor correlation between the level of the transcription of different genes and the relative abundance within the tissue of the corresponding proteins. Consequently, the information about a pathological process that can be derived at the level of gene transcription is incomplete. It is the high-throughput approach that defines and characterizes modern proteomics.

Despite the high complexity of components in urine, the urinary proteome is highly amenable to clinical research due to the wide availability of the samples, the non-invasive nature of collection and the possibility of repeat sampling. The urinary protein profile may reflect not only renal disease (O'Riordan, E. and Goligorsky, M. S. Curr Opin Nephrol Hypertens, 2005, 14(6):579-85), but also other diseases of the urinary tract, including bladder cancer. Thus, analysis of proteins in patient urine with a defined disease may provide a detailed knowledge of pathological process associated with the disease. Furthermore, the characterization of differences between asymptomatic and disease-associated urinary proteomes should provide markers for diagnosis and prognosis, and as potential targets for drug development.

Two-dimensional electrophoresis (2-DE) of proteins has been the conventional method for biomarker assessment in urological proteomics (Tantipaiboonwong, P. et al. Proteomics, 2005, 5(4):1140-9; Nabi, G. et al. Proteomics, 2005, 5(6):1729-33) and investigators have performed a systematic evaluation of sample preparation methods for gel-based human urinary proteomes (Thongboonkerd, V. et al. J Proteome Res, 2006, 5(1):183-91). A near-standard 2D map (Oh, J. et al. Proteomics, 2004, 4(11):3485-97) and high-resolution 2D gels (Pieper, R. et al. Proteomics, 2004, 4(4):1159-74) of urological tissues have contributed to the construction of a 2-D database (Wuhrer, M. et al. “Glycoproteomics based on tandem mass spectrometry of glycopeptides” J Chromatogr B Analyt Technol Biomed Life Sci, Oct. 16, 2006, Epub ahead of print; Zerefos, P. G. et al. Proteomics, 2006, 6(15):4346-55). The database also contains a listing 339 proteins detectable in urine, of which 124 have been identified. Using the urological 2-DE databases, pathologic subtypes of solid bladder tumor tissue specimens could be distinguished based on protein fingerprints (Wang, Y. et al. Glycobiology, 2006, 16(6):514-23; Liu, T. et al. J Proteome Res, 2005, 4(6):2070-80; Bunkenborg, J. et al. Proteomics, 2004, 4(2):454-65). Although a number of dysregulated proteins have been identified in a variety of tissue-based studies, it is disappointing that no reliable markers have been identified for transitional cell carcinoma, the most common type of bladder cancer. Through the proteomic study of a set of only 6 urine samples, Kageyama et al. (Clin Chem, 2004, 50(5):857-66) were able to identify a potential tumor marker, calreticulin, which is found in the urine of patients with bladder carcinoma. However, as is common in the search for cancer biomarkers, the authors used a differential display method and mass spectrometry to identify proteins that are increased in solid tumor tissues first, then subsequently monitored the presence of a reduced set of candidate biomarkers in urine samples. Unfortunately, in a larger cohort of patients, the diagnostic accuracy of calreticulin in urine was vulnerable to urinary tract infections (Kageyama, S. et al. Clin Chem, 2004, 50(5):857-66). Because none of the markers described to date have sufficient sensitivity and specificity for diagnostic use, the quest for identifying additional bladder cancer biomarkers continues.

Investigators have also applied gel-free methodologies to urine analysis. Pang et al. employed three different strategies to identify biomarkers related to acute inflammation (Pang, I. X. et al. J Proteome Res, 2002, 1(2):161-9), and Cutillas et al. used a 2D LC-MS/MS approach to generate a peptide profile of urine from patients with Dent's disease (Cutillas, P. R. et al. Clin Sci (Lond), 2003, 104(5):483-90). Most recently, Ru et al. used multi-dimensional protein identification technology (MudP1T), to identify 87 proteins in human urine (Ru, Q. C. et al. J Chromatogr A, 2006, 1111(2):166-74). While the necessity for a much smaller volume of urine would be desirable for diagnostic applications at home, the clinic, or laboratory, hundreds of milliliters of urine are typically used for gel-based analysis (Thongboonkerd, V. et al. J Proteome Res, 2006, 5(1):183-91; Oh, J. et al. Proteomics, 2004, 4(11):3485-97; Pieper, R. et al. Proteomics, 2004, 4(4):1159-74; Zerefos, P. G. et al. Proteomics, 2006, 6(15):4346-55; Pang, J. X. et al. J Proteome Res, 2002, 1(2):161-9). The pooling of samples from different individuals are typically observed in the published gel analysis of urines in order to increase the amount of proteins (Oh, J. et al. Proteomics, 2004, 4(11):3485-97; Zerefos, P. G. et al. Proteomics, 2006, 6(15):4346-55). By pooling samples, one is likely to lose the individual information of the intrinsic components present in urine from a single patient at a given time. Thus, a reliable and accurate profiling technique employing a small amount of urine is essential for detecting urinary proteomes and for marker identification. Low sample consumption also minimizes sample handling steps which reduce potential sample losses as well as analysis time.

Ideal cancer biomarkers should possess a high degree of sensitivity and specificity approaching 100%, and provide staging information complementary to that obtained from imaging studies or by invasive methods. An ideal biomarker should also provide predictive information concerning the natural history and response to treatment, and include the likelihood of disease recurrence or progression. Furthermore, the ideal biomarker should have technical characteristics that allow standardization and reproducibility (Montie et al., Urol. Clin. N. Amer., 1997, 45:247).

Current methods in the non-invasive detection and surveillance of bladder cancer via urine analysis include voided urine cytology (VUC) and some diagnostic urinary protein biomarkers; however, due to the poor sensitivity of VUC and high false-positive rates of currently available protein assays, detection of bladder cancer via urinalysis remains a challenge.

### BRIEF SUMMARY OF THE INVENTION

The present invention concerns biomarkers of bladder cancer in biological samples, such as human urine, and rapid, high-sensitivity methods to profile the N-linked glycoprotein component in naturally micturated human urine specimens. Con A affinity chromatography coupled to nanoflow liquid chromatography was utilized to separate the complex peptide mixture prior to a linear ion trap MS analysis. Of 186 proteins identified with high confidence by multiple analyses, 40% were secreted proteins, 18% membrane proteins and 14% extracellular proteins. In this study, the presence of several proteins appeared to be associated with the presence of bladder cancer, including alpha-1B-glycoprotein (A1BG) that was detected in all tumor-bearing patient samples but in none of the samples obtained from non-tumor bearing individuals. The combination of Con A affinity chromatography and nano-LC/MS/MS provides an initial investigation of N-glycoproteins in complex biological samples and facilitates the identification of potential biomarkers of bladder cancer in non-invasively obtained human urine.

Thus, the present invention relates to bladder cancer screening. The polypeptides listed in Table 4 herein constitute biomarkers for prognosis, diagnosis, and monitoring of bladder cancer. For example, one or more of the polypeptides listed in Table 4, or nucleic acids that encode the polypeptides, can be used to diagnose and monitor early stage and late stage bladder cancer. One or more polypeptides listed in Table 4, or nucleic acids that encode the polypeptides, can be used as a biomarker for bladder cancer before surgery and after relapse. These polypeptides and polynucleotides (referred to herein collectively as “biomarkers”, “bladder cancer biomarkers”, or grammatical variations thereof), and agents that bind to them, can be used to detect and monitor bladder cancer in male and female subjects.

Biomarkers of the invention include, but are not limited to, Alpha-1B-glycoprotein, Haptoglobin, Serotransferrin, Alpha-1-antitrypsin, Apolipoprotein(a), Epithelial-cadherin, Ig kappa chain V-ITT region SIE, Ig kappa chain V-III region B6, Alpha-1-microglobulin, Ig alpha-1 chain C region, Serum albumin, Zinc-alpha-2-glycoprotein, Prostaglandin-H2 D-isomerase, Ig lambda chain C regions, Ig kappa chain C region, CD59 glycoprotein, Polymeric-immunoglobulin receptor, Ig kappa chain V-II region Cum, Kininogen-1, CD44 antigen, Inter-alpha-inhibitor heavy chain 4, Uromodulin, Lysosomal alpha-glucosidase, LAMP-2, Arylsulfatase A, Attractin, and Kallikrein-1. In one embodiment, the biomarker is one or more polypeptides or nucleic acids encoding the polypeptides selected from Alpha-1B-glycoprotein, Haptoglobin, Serotransferrin, and Alpha-1-antitrypsin. In another embodiment, the biomarker is one or more polypeptides or encoding nucleic acids selected from the group consisting of Alpha-1B-glycoprotein, Haptoglobin, and Serotransferrin. In another embodiment, the biomarker is the polypeptides or nucleic acids encoding Alpha-1B-glycoprotein and/or Haptoglobin. In a preferred embodiment, the biomarker is the polypeptide Alpha-1B-glycoprotein, or nucleic acids encoding the polypeptide.

In one embodiment, bladder cancer is detected by screening for the presence or elevated levels of one or more polypeptides listed in Table 4, or their encoding polynucleotides, in urine samples. Optionally, the method further comprises verifying that the subject is suffering from the cancer detected (e.g., by assessing for the presence of one or more cancer symptoms, detecting additional cancer markers, biopsy, detecting the presence of the cancer through an imaging modality such as X-ray, CT, nuclear imaging (PET and SPECT), ultrasound, MRI), and/or treating the subject for the cancer detected (e.g., by surgery, chemotherapy, and/or radiation). In addition to a complete medical history and physical examination, diagnostic procedures for bladder cancer may include, for example, rectal or vaginal examination, cytoscopy (also called cystourethroscopy), intravenous pyelogram (IVP), laboratory tests, and the evaluation of the presence, absence, and/or concentration of markers of bladder cancer. Treatments for bladder cancer include, but are not limited to, surgery (transurethral resection, cystectomy, segmental cystectomy, or radical cystectomy), radiation therapy, chemotherapy, biological therapy (e.g., administration of bacilli Calmette-Guérin (BCG) into the bladder), phytodynamic therapy, and interferon.

The present invention also relates to kits for carrying out the methods of the invention.

In another aspect, the present invention relates to a device for the rapid detection of one or more of the biomarkers in urine. Preferably, the device is a lateral flow device. In one embodiment, the device comprises an application zone for receiving a sample of urine; a labeling zone containing a binding agent that binds to a biomarker in the sample; and a detection zone where biomarker-bound binding agent is retained to give a signal, wherein the signal given for a sample from a subject with a biomarker level lower than a threshold concentration is different from the signal given for a sample from a patient with a biomarker level equal to or greater than a threshold concentration.

In another aspect, the invention relates to a simple, rapid, reliable, accurate and cost effective test for bladder cancer biomarkers in voided urine, similar to currently available in-home pregnancy tests that could be used by subjects at home, in a physicians' office, or at a patient's bedside, e.g., at a health care facility. In one embodiment, the test is a method for detecting and, optionally, measuring, one or more biomarkers listed in Table 4 in urine, comprising: (a) obtaining a sample of urine from a subject; (b) contacting the sample with a binding agent that binds to any biomarker in the sample; (c) separating biomarker-bound binding agent; (d) detecting a signal associated with the separated binding agent from (c); and (e) comparing the signal detected in step (d) with a reference signal which corresponds to the signal given by a sample from a subject with a biomarker level equal to a threshold concentration. In one embodiment, the threshold concentration is between 0 ng/ml or pg/ml and an upper limit of ng/ml or pg/ml.

### BRIEF DESCRIPTION OF THE DRAWINGS

FIGS. 1A and 1B show chromatographic and sequencing data for urinary peptides. FIG. 1A shows a representative nanoLC/MS/MS base peak chromatogram, showing the detection of the peptide ions across the 40-min gradient separation. FIG. 1B shows MS/MS sequencing data of a peptide from uromodulin, identified in the eluted fraction of a bladder tumor-bearing patient urine sample.

FIGS. 2A-2C show the reproducibility of nanoLC/MS/MS of Con A-captured fractions from a human urine sample. The retention times of a +2 charged precursor mass of YFIDFVAR, from the protein kinnogen-1 from three independent runs were measured to be 22.84 (FIG. 2A), 22.73 (FIG. 2B), and 22.83 (FIG. 2C) minutes.

FIG. 3 shows the subcellular location of proteins identified in naturally micturated urine samples using a Con A lectin column and MS/MS analysis. A total of 186 urinary proteins were identified, with the majority of identified proteins being secreted (40%), membrane proteins (18%), and extracellular proteins (14%).

### DETAILED DESCRIPTION OF THE INVENTION

The present invention relates to a method for the diagnosis, prognosis, and monitoring of bladder cancer, such as early or late stage bladder cancer, by detecting in a urine sample from a subject at least one biomarker for bladder cancer identified herein, such as alpha-1B-glycoprotein, haptoglobin, serotransferrin, or alpha-1-antitrypsin. The biomarkers may be detected and, optionally, measured using an agent that detects or binds to the biomarker protein or an agent that detects or binds to encoding nucleic acids, such as antibodies specifically reactive with the biomarker protein or a portion thereof.

The feasibility of profiling a glycoprotein component of the naturally micturated urinary proteome was investigated, and an optimized analyses to compare the profile of a panel of urine samples obtained from patients with bladder cancer and non-malignant bladder conditions was applied. An immobilized alpha-mannose binding lectin, Concanavalin A (Con A), was used to enrich N-linked glycoproteins from human urine. The enriched glycoproteins were then digested with trypsin and analyzed with nano-LC/MS/MS. A total of 128 distinct N-linked glycoproteins were identified with high confidence by multiple analysis of as little as 10 ml of naturally micturated urine. As described in the Examples, the majority of identified proteins were secreted, or membranous proteins, and a subset of proteins was identified that was commonly excreted in urine from bladder cancer patients. The combinatorial approach of Con A affinity chromatography and nano-LC/MS/MS provides high sensitivity and with relatively moderate labor demands can greatly facilitate the identification of potential biomarkers of bladder cancer from non-invasively obtained human urine.

One aspect of the present invention concerns materials and methods for detecting and diagnosing bladder cancer and other urogenital related cancers. The polypeptides and/or nucleic acid molecules (e.g., DNA or mRNA) encoding the polypeptides listed in Table 4 can be used as molecular markers for bladder cancer. The biomarkers of the invention include, but are not limited to, Alpha-1B-glycoprotein, Haptoglobin, Serotransferrin, Alpha-1-antitrypsin, Apolipoprotein(a), Epithelial-cadherin, Ig kappa chain V-III region SIE, Ig kappa chain V-III region B6, Alpha-1-microglobulin, Ig alpha-1 chain C region, Serum albumin, Zinc-alpha-2-glycoprotein, Prostaglandin-H2 D-isomerase, Ig lambda chain C regions, Ig kappa chain C region, CD59 glycoprotein, Polymeric-immunoglobulin receptor, Ig kappa chain V-II region Cum, Kininogen-1, CD44 antigen, Inter-alpha-inhibitor heavy chain 4, Uromodulin, Lysosomal alpha-glucosidase, LAMP-2, Arylsulfatase A, Attractin, and Kallikrein-1. Protein and nucleotide sequences of biomarker proteins and nucleic acids encoding them can be found at numerous publicly available sequence databases including GenBank (see, for example, human Serotransferrin at accession numbers P02787 and NP_{—}001054; human Alpha-1B-glycoprotein at accession numbers NP_{—}570602 and AAH35719; human Haptoglobin at accession numbers AAA88080 and AAI21126; and human Alpha-1-antitrypsin at accession number CAA25838). In one embodiment, the biomarker is one or more polypeptides, and/or nucleic acids encoding the polypeptides, selected from one or more of Alpha-1B-glycoprotein, Haptoglobin, Serotransferrin, and Alpha-1-antitrypsin. In another embodiment, the biomarker is one or more polypeptides, and/or nucleic acids encoding the polypeptides, selected from Alpha-1B-glycoprotein, Haptoglobin, and Serotransferrin. In another embodiment, the biomarker is Alpha-1B-glycoprotein and/or Haptoglobin, and/or nucleic acids encoding these polypeptides. In a specific embodiment, the biomarker is the polypeptide Alpha-1B-glycoprotein, or nucleic acids encoding the polypeptide.

Cancer biomarkers (also called tumor biomarkers) are molecules such as hormones, enzymes, and immunoglobulins found in the body that are associated with cancer and whose measurement or identification is useful in patient diagnosis or clinical management. They can be products of the cancer cells themselves, or of the body in response to cancer or other conditions. Most cancer biomarkers are proteins. Some cancer biomarkers are seen only in a single type of cancer, while others can be detected in several types of cancer. As with other cancer biomarkers, the biomarkers described herein can be used for a variety of purposes, such as: screening a healthy population or a high risk population for the presence of bladder cancer; making a diagnosis of bladder cancer or of a specific type of bladder cancer; determining the prognosis of a subject; and monitoring the course in a subject in remission or while receiving surgery, radiation, chemotherapy, or other cancer treatment. Thus, urinary levels of these biomarkers can be used to detect and/or monitor the presence of bladder cancer throughout the course of disease and can be used in predicting therapeutic and prognostic outcome. For example, the biomarkers of the invention can be used to help corroborate the efficacy of chemopreventive drugs administered to treat, prevent, or delay recurrence of the disease.

One aspect of the invention concerns a method for detecting or diagnosing bladder cancer or other urogenital related cancer in a subject, comprising detecting the presence of and/or quantifying the level of at least one biomarker listed in Table 4 (such as alpha-1B-glycoprotein) in a sample, such as a urine sample, from the subject, wherein the presence of the biomarker, or a level (e.g., concentration) of the biomarker above a pre-determined threshold is indicative of bladder cancer or other urogenital cancer in the subject.

In one embodiment of a method of the invention, the detecting comprises: (a) contacting a biological sample with a binding agent (or binding agents) that binds the biomarker protein (or biomarker proteins) to form a complex (or complexes); and (b) detecting the complex(es); and optionally correlating the detected complex(es) to the amount of biomarker protein(s) in the sample, wherein the presence of one or more biomarkers, or the presence of elevated levels biomarker protein(s), is indicative of bladder cancer. In a specific embodiment, the binding agent is an aptamer or peptide or antibody. In a specific embodiment, the binding agent for detecting of step (b) further comprises a label linked or incorporated onto the agent. In one embodiment, the detecting comprising using ELISA-based immunoenzymatic detection. In another embodiment, the detecting comprises using Western blotting or radioimmunoassays (RIA).

In another embodiment, the biomarker protein (or proteins) is detected using electrophoretic, chromatographic, or spectroscopy methods, or a combination thereof. In one embodiment, proteins can be identified using 2-D gel electrophoresis of a sample followed by digestion of the separated proteins and mass spectrometry of peptides. In another embodiment, proteins or peptides can be identified by amino acid sequencing all or a portion of the molecule. In a specific embodiment, the mass spectrometry is tandem mass spectrometry (MS/MS). In a further embodiment, the mass spectrometry is carried out using matrix-assisted laser desorption/ionization (MALDI), such as MALDI-TOF. In a still further embodiment, proteins or peptides are identified using liquid chromatography (LC) methods, such as high pressure LC (HPLC).

Antibodies against numerous biomarker proteins of the invention are commercially available (e.g., anti-haptoglobulin (Sigma Life Science #H6395); anti-alpha-1-antitrypsin (Sigma Life Science #A0409-1VL); anti-alpha-1B-glycoprotein (Abnova Corporation #H00000001-M15); anti-serotransferrin (Lifespan Biosciences #LS-C45708)). Antibodies can also be readily prepared using standard and routine procedures known in the art.

Optionally, the methods of the invention further comprise detecting and/or quantifying one or more additional biomarkers of bladder cancer or other urogenital cancer and/or one or more additional biomarkers of a different cancer type in the same urine sample, a different urine sample, or a same or different biological sample (e.g., serum, plasma, whole blood, tissue (e.g., biopsy), exfoliated urothelial cells or bladder cancer cells) obtained from the same subject, before, during, or after said detecting of the biomarker(s) of the invention is carried out on the sample. In this way, one or more biomarkers of the invention can be used as part of a panel of biomarkers utilized in surveillance protocols for detecting bladder cancer or other urogenital cancer and, optionally, other cancers. For example, a panel of 2, 3, 4, 5, 6, 7, 8, 9, or 10 or more markers could be utilized.

In some embodiments, the detecting is performed at several time points or intervals, as part of monitoring of the subject before, during, or after treatment of the cancer.

Optionally, the methods of the invention further comprise comparing the level of one or more bladder cancer biomarkers in a urine or other biological sample with the level of biomarker present in a normal control sample, wherein a higher level of biomarker in the sample as compared to the level in the normal control sample is indicative of the presence of cancer.

In some embodiments, the subject exhibits no symptoms of cancer at the time the detecting of the bladder cancer biomarker(s) is carried out. In other embodiments, the subject exhibits one or more symptoms of cancer, such as bladder cancer, at the time the detecting of bladder cancer biomarker(s) is carried out. For example, with respect to bladder cancer, the one or more symptoms of bladder cancer can include, but are not limited to, visible blood in the urine, hematuria (the presence of microscopic red blood cells in the urine), painful urination, urgency (frequently feeling the need to urinate without results), frequent urination, and pelvic or flank pain.

The subject invention also concerns methods for prognostic evaluation of a subject having, or suspected of having, cancer such as bladder cancer, comprising: a) determining the level of one or more cancer biomarkers of the present invention (for example, one of the biomarkers listed in Table 4) in a biological sample obtained from the subject; b) comparing the level determined in step (a) to a level or range of the one or more cancer biomarkers known to be present in a biological sample obtained from a normal subject that does not have cancer; and c) determining the prognosis of the subject based on the comparison of step (b), wherein a high level of the one or more cancer biomarkers in step (a) indicates a more aggressive form of cancer and, therefore, a poor prognosis. In one embodiment, the biomarker is one or more polypeptides, and/or nucleic acids encoding the polypeptides, selected from one or more of Alpha-1B-glycoprotein, Haptoglobin, Serotransferrin, and Alpha-1-antitrypsin. In another embodiment, the biomarker is one or more polypeptides, and/or nucleic acids encoding the polypeptides, selected from Alpha-1B-glycoprotein, Haptoglobin, and Serotransferrin. In another embodiment, the biomarker is Alpha-1B-glycoprotein and/or Haptoglobin, and/or nucleic acids encoding these polypeptides. In a preferred embodiment, the biomarker is the polypeptide Alpha-1B-glycoprotein, or nucleic acids encoding the polypeptide.

The terms “detecting” or “detect” include assaying or otherwise establishing the presence or absence of the target bladder cancer biomarker (one or more encoding nucleic acid sequences or gene products (polypeptides) listed in Table 4 herein, such as alpha-1B-glycoprotein), subunits thereof, or combinations of agent bound targets, and the like, or assaying for, interrogating, ascertaining, establishing, or otherwise determining one or more factual characteristics of bladder cancer, metastasis, stage, or similar conditions. The term encompasses diagnostic, prognostic, and monitoring applications for one or more of the bladder cancer biomarkers in Table 4 (such as alpha-1B-glycoprotein) and, optionally, other cancer biomarkers. The term encompasses quantitative, semi-quantitative, and qualitative detection methodologies. In embodiments of the invention involving detection of one or more polypeptides (as opposed to nucleic acid molecules encoding the polypeptides), the detection method may be, for example, an ELISA-based method. Preferably, in the various embodiments of the invention, the detection method provides an output (i.e., readout or signal) with information concerning the presence, absence, or amount of the bladder cancer biomarker(s) in a urine sample from a subject. For example, the output may be qualitative (e.g., “positive” or “negative”), or quantitative (e.g., a concentration such as nanograms per milliliter).

In one embodiment, the invention relates to a method for detecting bladder cancer in a subject by quantitating one or more bladder cancer biomarker polypeptides listed in Table 4 (such as alpha-1B-glycoprotein), or their encoding nucleic acids (DNA or RNA), in a biological sample, such as a urine sample, from the subject, comprising (a) contacting (reacting) the sample with an antibody specific for the biomarker polypeptide(s) which is directly or indirectly labeled with a detectable substance; and (b) detecting the detectable substance. In one embodiment, the biomarker is one or more polypeptides, and/or nucleic acids encoding the polypeptides, selected from one or more of Alpha-1B-glycoprotein, Haptoglobin, Serotransferrin, and Alpha-1-antitrypsin. In another embodiment, the biomarker is one or more polypeptides, and/or nucleic acids encoding the polypeptides, selected from Alpha-1B-glycoprotein, Haptoglobin, and Serotransferrin. In another embodiment, the biomarker is Alpha-1B-glycoprotein and/or Haptoglobin, and/or nucleic acids encoding these polypeptides. In a preferred embodiment, the biomarker is the polypeptide Alpha-1B-glycoprotein, or nucleic acids encoding the polypeptide.

In one embodiment, the invention relates to a method for diagnosing and/or monitoring cancer in a subject by quantitating one or more bladder cancer biomarker polypeptides listed in Table 4 in a biological sample, such as a urine sample, from the subject, comprising (a) reacting the sample with an antibody or antibodies specific for the biomarker or biomarkers which are directly or indirectly labeled with a detectable substance; and (b) detecting the detectable substance. In one embodiment, the biomarker is one or more polypeptides, and/or nucleic acids encoding the polypeptides, selected from one or more of Alpha-1B-glycoprotein, Haptoglobin, Serotransferrin, and Alpha-1-antitrypsin. In another embodiment, the biomarker is one or more polypeptides, and/or nucleic acids encoding the polypeptides, selected from Alpha-1B-glycoprotein, Haptoglobin, and Serotransferrin. In another embodiment, the biomarker is Alpha-1B-glycoprotein and/or Haptoglobm, and/or nucleic acids encoding these polypeptides. In a preferred embodiment, the biomarker is the polypeptide Alpha-1B-glycoprotein, or nucleic acids encoding the polypeptide.

Some embodiments of the methods of the invention involve (a) contacting a biological sample, such as a urine sample, from a subject with an antibody or antibodies specific for the biomarker or biomarker polypeptides listed in Table 4 (such as alpha-1B-glycoprotein) which are directly or indirectly labeled with an enzyme; (b) adding a substrate for the enzyme wherein the substrate is selected so that the substrate, or a reaction product of the enzyme and substrate, forms fluorescent complexes; (c) quantitating the biomarker(s) in the sample by measuring fluorescence of the fluorescent complexes; and (d) comparing the quantitated levels to that of a standard. In one embodiment, the biomarker is one or more polypeptides, and/or nucleic acids encoding the polypeptides, selected from one or more of Alpha-1B-glycoprotein, Haptoglobin, Serotransferrin, and Alpha-1-antitrypsin. In another embodiment, the biomarker is one or more polypeptides, and/or nucleic acids encoding the polypeptides, selected from Alpha-1B-glycoprotein, Haptoglobin, and Serotransferrin. In another embodiment, the biomarker is Alpha-1B-glycoprotein and/or Haptoglobin, and/or nucleic acids encoding these polypeptides. In a preferred embodiment, the biomarker is the polypeptide Alpha-1B-glycoprotein, or nucleic acids encoding the polypeptide.

A specific embodiment of the invention comprises:

A method for detecting a urogenital cancer comprising (a) incubating a biological sample with a first antibody specific for at least one bladder cancer biomarker polypeptide listed in Table 4 (such as alpha-1B-glycoprotein) which is directly or indirectly labeled with a detectable substance, and a second antibody specific for the biomarker polypeptide which is immobilized;

(b) separating the first antibody from the second antibody to provide a first antibody phase and a second antibody phase;

(c) detecting the detectable substance in the first or second antibody phase thereby quantitating the biomarker in the urine sample; and

(d) comparing the quantitated biomarker with a standard.

A standard used in a method of the invention may correspond to biomarker levels obtained for samples from healthy control subjects, from subjects with benign disease (e.g., benign urogenital disease), subjects with early stage bladder cancer, or from other samples of the subject. Increased levels of a bladder cancer biomarker as compared to the standard may be indicative of bladder cancer, such as early or late stage bladder cancer. In one embodiment, the biomarker is one or more polypeptides, and/or nucleic acids encoding the polypeptides, selected from one or more of Alpha-1B-glycoprotein, Haptoglobin, Serotransferrin, and Alpha-1-antitrypsin. In another embodiment, the biomarker is one or more polypeptides, and/or nucleic acids encoding the polypeptides, selected from Alpha-1B-glycoprotein, Haptoglobin, and Serotransferrin. In another embodiment, the biomarker is Alpha-1B-glycoprotein and/or Haptoglobin, and/or nucleic acids encoding these polypeptides. In a preferred embodiment, the biomarker is the polypeptide Alpha-1B-glycoprotein, or nucleic acids encoding the polypeptide.

The invention also contemplates using the methods, devices, and kits described herein in conjunction with one or more additional biomarkers for cancer. Therefore, the invention contemplates a method for analyzing a biological sample, such as a urine sample, for the presence of a bladder cancer biomarker of the invention (a polypeptide or encoding nucleic acid molecule listed in Table 4, such as alpha-1B-glycoprotein) and analyzing the same sample, or another biological sample from the same subject, for other markers that are specific indicators of a cancer such as bladder cancer. The one or more additional markers may be detected before, during, and/or after detection of the one or more bladder cancer biomarkers of the invention is carried out. The methods, devices, and kits described herein may be modified by including agents to detect the additional markers, or nucleic acids encoding the markers.

In one embodiment, the additional biomarker is one or more additional diagnostic and/or prognostic biomarkers for bladder cancer selected from complement factor H-related protein, nuclear matrix protein 22 (NMP22), survivin, cytokeratins 8, 18, 19, and/or 20, hyaluronic acid (HA), hyaluronidase (HAase), microsatellite mutation, mucin-like protein, carcinoembryonic antigen (CEA), chromosome 9 deletion, p53 mutation, heat shock protein 70 (HSP 70), tumor associated antigen 138 (T138), blood group antigens (ABO), oncogenes (such as c-H-ras, c-myc, c-erb B2, or mdm-2), cell cycle regulators (such as Rb, p53, p21, or p27), proliferation-associated antigens (such as Ki-67, PCNA, or MCM), vessel density, thrombospondin-1, cell adhesion molecules (such as cadherins, integrins, or the Ig-super family), extracellular matrix proteases (such as laminin-P1, cathepsin D, U-PA, matrix metalloproteases, and matrix metalloprotease inhibitors), growth factor receptors (such as EGFR), peptide growth factors (such as EGF, FGF, TGF-alpha, TGF-beta, or VEGF), apoptosis markers (such as Bcl2, bax, Fas, or Fas-L), CD44, and BLCA-4 (Black et al., J. Clin. Oncol., 2006, 24:5528-5535; Syrigos et al., Hybridoma and Hybridomics, 2004, 23(6):335-342; Kausch and Bohle, European Urology, 2002, 41:1529; Stein et al., Clinical Urology, 1998, 160 (3-I):645-659; Mao et al., Science, 1996, 271(5249):659-662; Mao L., J. Cell. Biochem., 1997, 63(S25):191-196; Van Le et al., Clinical Cancer Research, 2004, 10:184-1391; Arisan S., Turkish Journal of Cancer, 2003, 33(4):171-176; Lanbein et al., Technology in Cancer Research and Treatment, 2006, 5(1):67-71; Ziaee et al., Urology Journal, 2006, 3(3):150-153, which are each incorporated herein by reference in their entirety).

Other cancer markers that may be used in conjunction with the invention include, but are not limited to: alpha fetoprotein (AFP), e.g., for pancreatic, kidney, ovarian, cervical, and testicular cancers; carcinogenic embryonic antigen (CEA), e.g., for lung, pancreatic, kidney, breast, uterine, liver, gastric, and colorectal cancers; carbohydrate antigen 15-3 (CA15-3), e.g., for lung, pancreatic, breast, ovarian, and liver cancers; carbohydrate antigen 19-9 (CA19-9), e.g., for lung, ovarian, uterine, liver, gastric, colorectal, and bile duct cancers; cancer antigen 125 (CA125), e.g., for lung, pancreas, breast, ovarian, cervical, uterine, liver, gastric, and colorectal cancers; free prostate specific antigen and prostate specific antigen-alpha(1) (PSA), for prostate cancer; free prostate specific antigen (PSAF), for prostate and colorectal cancers; prostate specific antigen-alpha(1)antichymotrypsin complex (PSAC), for prostate cancer; prostatic acid phosphatase (PAP), for prostate cancer; human thyroglobulin (hTG), for thyroid cancer or Wilm's tumor; human chorionic gonadaotropin beta (hCGb), e.g., for lung, pancreatic, kidney, ovarian, uterine, testicular, liver, colorectal, bladder, and brain cancers; ferritin (Ferr), e.g., for lung cancer, testicular cancer, cancer of the larynx, Burkitt's lymphoma, neuroblastoma, and leukemia; neuron specific enolase (NSE), for lung cancer, thyroid cancer, Wilm's tumor, and neuroblastoma; interleukin 2 (IL-2), for kidney cancer and multiple myeloma; interleukin 6 (IL-6), for kidney cancer, breast cancer, ovarian cancer, and multiple myeloma; beta 2 microglobulin (B2M), for kidney cancer, ovarian cancer, prostate cancer, leukemia, multiple myeloma, and lymphoma; and/or alpha 2 microglobulin (A2M), for prostate cancer. The selection of a biological sample (such as whole blood, serum, plasma, or urine) in which the aforementioned cancer markers are diagnostic and/or prognostic can be readily determined by those skilled in the art.

In another embodiment, the invention incorporates one or more of the following additional cancer marker(s): alpha-fetoprotein (AFP), e.g., for hepatocellular cancer; AFP and b-HCG (the combination), e.g., for testicular or ovarian cancer; beta₂-microglobulin (B₂M), e.g., for tumor burden in patients with multiple myeloma; calcitonin, e.g., for early medullary carcinoma of the thyroid; cancer antigen 19-9 (CA19-9), e.g., for gastric and/or pancreatic cancer; CA-125, e.g., for ovarian cancer and/or endometrial cancer; CA15-3, CA27-29, and/or Truquant RIA, e.g., for advanced breast cancer; carcinoembryonic antigen (CEA), e.g., for colorectal cancer or hepatocellular cancer; estrogen and/or progesterone receptors, e.g., for primary or metastatic breast cancer; HER-2-Neu, e.g., for primary or metastatic breast cancer; human chorionic gonadotropin (HCG), e.g., for trophoblastic tumors of the ovaries and testes or for choriocarcinomas; neuron-specific enolase (NSE), e.g., for small cell cancer of the lung; prostatic acid phosphatase (PAP), e.g., for prostate cancer; prostate-specific antigen (PSA), e.g., for prostate cancer; and/or throglobulin, e.g., for follicular or papillary carcinoma of the thyroid.

As indicated above, the present invention provides a method for detecting, diagnosing, or for monitoring the prognosis of bladder cancer in a subject by detecting a bladder cancer biomarker of the invention in a biological sample from the subject. In one embodiment, the method comprises contacting the sample with an antibody specific for the biomarker polypeptide which is directly or indirectly labeled with a detectable substance, and detecting the detectable substance.

The methods, devices, and kits of the invention can be used for the detection of either an over-abundance or an under-abundance of one or more bladder cancer biomarkers relative to a non-disorder state or the presence of a modified (e.g., less than full length) bladder cancer biomarker which correlates with a disorder state (e.g., bladder cancer), or a progression toward a disorder state. The methods described herein can also be used to evaluate the probability of the presence of malignant or pre-malignant cells. Such methods can be used to detect tumors, quantitate their growth, and assist in the diagnosis and prognosis of urogenital cancer such as bladder cancer. The methods can also be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods, devices, and kits of the invention can be used in the diagnosis of early stage bladder cancer (e.g., when the subject is asymptomatic) and for monitoring and evaluating the prognosis of bladder cancer disease progression and mortality. Depending upon the particular bladder cancer biomarker of the invention, increased levels or decreased levels of detected biomarker in a urine sample compared to a standard (e.g., levels for normal or benign disorders) may be indicative of advanced disease stage, large residual tumor, and/or increased risk of disease progression and mortality.

The terms “sample”, “biological sample”, and the like refer to a type of material known to or suspected of expressing or containing a biomarker of cancer, such as urine. The test sample can be used directly as obtained from the source or following a pretreatment to modify the character of the sample. The sample can be derived from any biological source, such as tissues or extracts, including cells (e.g., tumor cells) and physiological fluids, such as, for example, whole blood, plasma, serum, peritoneal fluid, ascites, and the like. The sample can be obtained from animals, preferably mammals, most preferably humans. The sample can be pretreated by any method and/or can be prepared in any convenient medium that does not interfere with the assay. The sample can be treated prior to use, such as preparing plasma from blood, diluting viscous fluids, applying one or more protease inhibitors to samples such as urine, and the like. Sample treatment can involve filtration, distillation, extraction, concentration, inactivation of interfering components, the addition of reagents, and the like.

The presence of a bladder cancer biomarker of the invention (polypeptides listed in Table 4 (such as alpha-1B-glycoprotein) or nucleic acids (DNA or RNA) encoding the polypeptides) may be detected in human or non-human mammalian urine.

In several embodiments of the invention, the method described herein is adapted for diagnosing and monitoring bladder cancer by quantitating a biomarker of the invention in urine samples from a subject. Preferably, the amount of biomarker quantitated in a urine sample from a subject being tested is compared to levels quantitated for another sample or an earlier sample from the subject, or levels quantitated for a control sample. Levels for control samples from healthy subjects may be established by prospective and/or retrospective statistical studies. Healthy subjects who have no clinically evident disease or abnormalities may be selected for statistical studies. Diagnosis may be made by a finding of statistically different levels of one or more biomarkers compared to a control sample or previous levels quantitated for the same subject.

The bladder cancer biomarkers of the invention include all homologs, naturally occurring allelic variants, isoforms and precursors of the human or non-human molecules. In general, naturally occurring allelic variants of human biomarkers will share significant sequence homology (70-90%) to other sequences. Allelic variants may contain conservative amino acid substitutions or will contain a substitution of an amino acid from a corresponding position in a homologue.

The terms “subject” and “patient” are used interchangeably herein to refer to a warm-blooded animal, such as a mammal, which may be afflicted with cancer such as bladder cancer. The subject may be male or female. The term includes dogs, cats, and horses. The term also includes primates such as apes, chimps, monkeys, and humans.

Agents that are capable of detecting bladder cancer biomarkers of the invention in the urine samples of subjects are those that interact or bind with the polypeptide or the nucleic acid molecule encoding polypeptide. Examples of such agents (also referred to herein as binding agents) include, but are not limited to, antibodies or fragments thereof that bind the polypeptide, polypeptide binding partners, and nucleic acid molecules that hybridize to the nucleic acid molecules encoding the polypeptides. Preferably, the binding agent is labeled with a detectable substance (e.g., a detectable moiety). The binding agent may itself function as a label.

### Biomarker Antibodies

Antibodies specific for bladder cancer biomarkers of the invention (such as alpha-1B-glycoprotein) that are used in the methods, devices, and kits of the invention may be obtained from scientific or commercial sources. Alternatively, the isolated native polypeptides or recombinant polypeptides may be utilized to prepare antibodies, monoclonal or polyclonal antibodies, and immunologically active fragments (e.g., a Fab or (Fab)₂ fragment), an antibody heavy chain, an antibody light chain, humanized antibodies, a genetically engineered single chain Fᵥ molecule (Ladle et al., U.S. Pat. No. 4,946,778), or a chimeric antibody, for example, an antibody which contains the binding specificity of a murine antibody, but in which the remaining portions are of human origin. Antibodies, including monoclonal and polyclonal antibodies, fragments and chimeras, may be prepared using methods known to those skilled in the art. Preferably, antibodies used in the methods of the invention are reactive against biomarkers of the invention if they bind with a Kₐ of greater than or equal to 10⁷ M. In a sandwich immunoassay of the invention, mouse polyclonal antibodies and rabbit polyclonal antibodies can be utilized, for example.

In order to produce monoclonal antibodies, a host mammal is inoculated with a protein or peptide representing a bladder cancer biomarker of the invention and then boosted. Spleens are collected from inoculated mammals a few days after the final boost. Cell suspensions from the spleens are fused with a tumor cell in accordance with the general method described by Kohler and Milstein (Nature, 1975, 256:495-497). In order to be useful, a peptide fragment must contain sufficient amino acid residues to define the epitope of the biomarker molecule being detected.

If the fragment is too short to be immunogenic, it may be conjugated to a carrier molecule. Some suitable carrier molecules include keyhole limpet hemocyanin and bovine serum albumin. Conjugation may be carried out by methods known in the art. One such method is to combine a cysteine residue of the fragment with a cysteine residue on the carrier molecule. The peptide fragments may be synthesized by methods known in the art. Some suitable methods are described by Stuart and Young in “Solid Phase Peptide Synthesis,” Second Edition, Pierce Chemical Company (1984).

Purification of the antibodies or fragments can be accomplished by a variety of methods known to those skilled in the art including, precipitation by ammonium sulfate or sodium sulfate followed by dialysis against saline, ion exchange chromatography, affinity or immunoaffinity chromatography as well as gel filtration, zone electrophoresis, etc. (Goding in, Monoclonal Antibodies: Principles and Practice, 2d ed., pp. 104-126, Orlando, Fla., Academic Press). It is preferable to use purified antibodies or purified fragments of the antibodies having at least a portion of a biomarker binding region, including such as Fv, F(ab)₂, Fab fragments (Harlow and Lane, 1988, Antibody Cold Spring Harbor) for the detection of the biomarker(s) in the urine of bladder cancer patients or those at risk.

For use in detection and/or monitoring of cancer, the purified antibodies can be covalently attached, either directly or via linker, to a compound which serves as a reporter group to permit detection of the presence of the biomarker. A variety of different types of substances can serve as the reporter group, including but not limited to enzymes, dyes, radioactive metal and non-metal isotopes, fluorogenic compounds, fluorescent compounds, etc. Methods for preparation of antibody conjugates of the antibodies (or fragments thereof) of the invention useful for detection, monitoring are described in U.S. Pat. Nos. 4,671,958; 4,741,900 and 4,867,973.

In one aspect of the invention, preferred binding epitopes may be identified from a known biomarker gene sequence and its encoded amino acid sequence and used to generate antibodies to the biomarker with high binding affinity. Also, identification of binding epitopes on the biomarker polypeptide can be used in the design and construction of preferred antibodies. For example, a DNA encoding a preferred epitope on a biomarker polypeptide may be recombinantly expressed and used to select an antibody which binds selectively to that epitope. The selected antibodies then are exposed to the sample under conditions sufficient to allow specific binding of the antibody to the specific binding epitope on the biomarker and the amount of complex formed then detected. Specific antibody methodologies are well understood and described in the literature. A more detailed description of their preparation can be found, for example, in Practical Immunology, Butt, W. R., ed., Marcel Dekker, New York, 1984.

The present invention also contemplates the detection of biomarker antibodies. Thus, detection of antibodies to the biomarkers of the invention in urine of a subject may enable the diagnosis of bladder cancer and is also contemplated within the scope of the invention.

### Protein Binding Assays

Antibodies specifically reactive with the biomarkers listed in Table 4 or their derivatives, such as enzyme conjugates or labeled derivatives, may be used to the detect biomarkers in various biological samples, for example they may be used in any known immunoassays which rely on the binding interaction between an antigenic determinant of a protein and the antibodies. Examples of such assays are radioimmunoassays, enzyme immunoassay (e.g., ELISA), immunofluorescence, immunoprecipitation, latex agglutination, hemagglutination, and histochemical tests.

An antibody specific for a biomarker of the invention can be labeled with a detectable substance and localized in biological samples such as urine based upon the presence of the detectable substance. Examples of detectable substances include, but are not limited to, the following radioisotopes (e.g., ³H, ¹⁴C, ³⁵S, ¹²⁵I, ¹³¹I) fluorescent labels (e.g., FITC, rhodamine, lanthanide phosphors), luminescent labels such as luminol; enzymatic labels (e.g., horseradish peroxidase, beta-galactosidase, luciferase, alkalline phosphatase, acetylcholinestease), biotinyl groups (which can be detected by marked avidin, e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). Indirect methods may also be employed in which the primary antigen-antibody reaction is amplified by the introduction of a second antibody, having specificity for the antibody reactive against the biomarker. By way of example, if the antibody having specificity against a biomarker is a rabbit IgG antibody, the second antibody may be goat anti-rabbit gamma-globulin labeled with a detectable substance.

Methods for conjugating or labeling the antibodies discussed above may be readily accomplished by one of ordinary skill in the art. (See, for example, Imman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem., 1988, 171:1-32, regarding methods for conjugating or labeling the antibodies with an enzyme or ligand binding partner).

Time-resolved fluorometry may be used to detect a signal. For example, the method described in Christopoulos T. K. and Diamandis E. P., Anal. Chem., 1992:64:342-346 may be used with a conventional time-resolved fluorometer.

Therefore, in accordance with an embodiment of the invention, a method is provided wherein an antibody to a bladder cancer biomarker of the invention is labeled with an enzyme, a substrate for the enzyme is added wherein the substrate is selected so that the substrate, or a reaction product of the enzyme and substrate, forms fluorescent complexes with a lanthanide metal. A lanthanide metal is added and the biomarker is quantitated in the sample by measuring fluorescence of the fluorescent complexes. The antibodies specific for the biomarkers may be directly or indirectly labeled with an enzyme. Enzymes are selected based on the ability of a substrate of the enzyme, or a reaction product of the enzyme and substrate, to complex with lanthanide metals such as europium and terbium. Examples of suitable enzymes include alkalline phosphatase and beta-galactosidase. Preferably, the enzyme is akline phosphatase. The biomarker antibodies may also be indirectly labeled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. Preferably the antibodies are biotinylated, and the enzyme is coupled to streptavidin.

In an embodiment of the method, antibody bound to a biomarker of the invention in a sample is detected by adding a substrate for the enzyme. The substrate is selected so that in the presence of a lanthanide metal (e.g., europium, terbium, samarium, and dysprosium, preferably europium and terbium), the substrate or a reaction product of the enzyme and substrate, forms a fluorescent complex with the lanthanide metal. Examples of enzymes and substrates for enzymes that provide such fluorescent complexes are described in U.S. Pat. No. 5,312,922 to Diamandis. By way of example, when the antibody is directly or indirectly labeled with alkalline phosphatase, the substrate employed in the method may be 4-methylumbeliferyl phosphate, or 5-fluorpsalicyl phosphate. The fluorescence intensity of the complexes is typically measured using a time-resolved fluorometer, e.g., a CyberFluor 615 Immoanalyzer (Nordion International, Kanata Ontario).

The sample, antibody specific for the biomarker, or the biomarker itself, may be immobilized on a carrier. Examples of suitable carriers are agarose, cellulose, dextran, Sephadex, Sepharose, liposomes, carboxymethyl cellulose polystyrene, filter paper, ion-exchange resin, plastic film, plastic tube, glass beads, polyamine-methyl vinyl ether-maleic acid copolymer, amino acid copolymer, ethylene-maleic acid copolymer, nylon, silk, etc. The carrier may be in the shape of, for example, a tube, test plate, well, beads, disc, sphere, etc. The immobilized antibody may be prepared by reacting the material with a suitable insoluble carrier using known chemical or physical methods, for example, cyanogen bromide coupling.

In accordance with an embodiment, the present invention provides a mode for determining the presence and, optionally, the amount of bladder cancer biomarker in an appropriate sample such as urine by measuring the biomarker(s) by immunoassay. It will be evident to a skilled artisan that a variety of immunoassay methods can be used to measure the biomarkers of the invention. In general, a biomarker immunoassay method may be competitive or noncompetitive. Competitive methods typically employ an immobilized or immobilizable antibody to the biomarker (anti-biomarker such as anti-alpha-1B-glycoprotein) and a labeled form of the biomarker (such as labeled alpha-1B-glycoprotein). Sample biomarker and labeled biomarker compete for binding to anti-biomarker. After separation of the resulting labeled biomarker that has become bound to anti-biomarker (bound fraction) from that which has remained unbound (unbound fraction), the amount of the label in either bound or unbound fraction is measured and may be correlated with the amount of biomarker in the biological sample in any conventional manner, e.g., by comparison to a standard curve.

Preferably, a noncompetitive method is used for the determination of one or more biomarkers of the invention, with the most common method being the “sandwich” method. In this assay, two anti-biomarker antibodies, such as two anti-alpha-1B-glycoprotein antibodies, are employed. One of the anti-biomarker antibodies is directly or indirectly labeled (also referred to as the “detection antibody”) and the other is immobilized or immobilizable (also referred to as the “capture antibody”). The capture and detection antibodies can be contacted simultaneously or sequentially with the biological sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally, it is measured in the capture antibody phase since it comprises the biomarker bound by (“sandwiched” between) the capture and detection antibodies.

In a typical two-site immunometric assay for a biomarker, one or both of the capture and detection antibodies are polyclonal antibodies. The label used in the detection antibody can be selected from any of those known conventionally in the art. As with other embodiments of the protein detection assay, the label can be an enzyme or a chemiluminescent moiety, for example, or a radioactive isotope, a fluorophore, a detectable ligand (e.g., detectable by a secondary binding by a labeled binding partner for the ligand), and the like. Preferably, the antibody is labeled with an enzyme that is detected by adding a substrate that is selected so that a reaction product of the enzyme and substrate forms fluorescent complexes. The capture antibody is selected so that it provides a mode for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody can comprise an antibody covalently or noncovalently attached to a solid phase such as a magnetic particle, a latex particle, a microtiter multi-well plate, a bead, a cuvette, or other reaction vessel. An example of an immobilizable capture antibody is an antibody that has been chemically modified with a ligand moiety, e.g., a hapten, biotin, or the like, and that can be subsequently immobilized by contact with an immobilized form of a binding partner for the ligand, e.g., an antibody, avidin, or the like. In an embodiment, the capture antibody can be immobilized using a species specific antibody for the capture antibody that is bound to the solid phase.

A particular sandwich immunoassay method of the invention employs two antibodies reactive against a biomarker of the invention, a second antibody having specificity against an antibody reactive against the biomarker labeled with an enzymatic label, and a fluorogenic substrate for the enzyme. In an embodiment, the enzyme is alkalline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb³⁺ fluorescence in a time-resolved mode. Fluorescence intensity is typically measured using a time-resolved fluorometry as described herein.

The above-described immunoassay methods and formats are intended to be exemplary and are not limiting since, in general, it will be understood that any immunoassay method or format can be used in the present invention.

The protein detection methods, devices, and kits of the invention can utilize nanowire sensor technology (Zhen et al., Nature Biotechnology, 2005, 23(10):1294-1301; Lieber et al., Anal. Chem 2006, 78(13):4260-4269, which are incorporated herein by reference) or microcantilever technology (Lee et al., Biosens. Bioelectron, 2005, 20(10):2157-2162; Wee et al., Biosens. Bioelectron., 2005, 20(10):1932-1938; Campbell and Mutharasan, Biosens. Bioelectron., 2005, 21(3):462-473; Campbell and Mutharasan, Biosens. Bioelectron., 2005, 21(4):597-607; Hwang et al., Lab chip, 2004, 4(6):547-552; Mukhopadhyay et al., Nano. Lett., 2005, 5(12):2835-2388, which are incorporated herein by reference) for detection of one or more biomarkers of the invention in samples. In addition, Huang et al. describe a prostate specific antigen immunoassay on a commercially available surface plasmon resonance biosensor (Biosens. Bioelectron., 2005, 21(3):483-490, which is incorporated herein by reference) which may be adapted for detection of one or more biomarkers of the invention. High-sensitivity miniaturized immunoassays may also be utilized for detection of the biomarkers (Cesaro-Tadic et al., Lab chip, 2004, 4(6):563-569; Zimmerman et al., Biomed. Microdevices, 2005, 7(2):99-110, which are incorporated herein by reference).

### Nucleic Acids

Nucleic acids including naturally occurring nucleic acids, oligonucleotides, antisense oligonucleotides, and synthetic oligonucleotides that hybridize to the nucleic acid encoding biomarker polypeptides of the invention, are useful as agents to detect the presence of biomarkers of the invention in the biological samples of cancer patients or those at risk of cancer, preferably in the urine of bladder cancer patients or those at risk of bladder cancer. The present invention contemplates the use of nucleic acid sequences corresponding to the coding sequence of biomarkers of the invention and to the complementary sequence thereof, as well as sequences complementary to the biomarker transcript sequences occurring further upstream or downstream from the coding sequence (e.g., sequences contained in, or extending into, the 5′ and 3′ untranslated regions) for use as agents for detecting the expression of biomarkers of the invention in biological samples of cancer patients, or those at risk of cancer, preferably in the urine of bladder cancer patients or those at risk of bladder cancer.

The preferred oligonucleotides for detecting the presence of biomarkers of the invention in biological samples are those that are complementary to at least part of the cDNA sequence encoding the biomarker. These complementary sequences are also known in the art as “antisense” sequences. These oligonucleotides may be oligoribonucleotides or oligodeoxyribonucleotides. In addition, oligonucleotides may be natural oligomers composed of the biologically significant nucleotides, i.e., A (adenine), dA (deoxyadenine), G (guanine), dG (deoxyguanine), C (cytosine), dC (deoxycytosine), T (thymine) and U (uracil), or modified oligonucleotide species, substituting, for example, a methyl group or a sulfur atom for a phosphate oxygen in the inter-nucleotide phosohodiester linkage. Additionally, these nucleotides themselves, and/or the ribose moieties may be modified.

The oligonucleotides may be synthesized chemically, using any of the known chemical oligonucleotide synthesis methods well described in the art. For example, the oligonucleotides can be prepared by using any of the commercially available, automated nucleic acid synthesizers. Alternatively, the oligonucleotides may be created by standard recombinant DNA techniques, for example, inducing transcription of the noncoding strand. The DNA sequence encoding the biomarker may be inverted in a recombinant DNA system, e.g., inserted in reverse orientation downstream of a suitable promoter, such that the noncoding strand now is transcribed.

Although any length oligonucleotide may be utilized to hybridize to a nucleic acid encoding a biomarker polypeptide, oligonucleotides typically within the range of 8-100 nucleotides are preferred. Most preferable oligonucleotides for use in detecting biomarkers in urine samples are those within the range of 15-50 nucleotides.

The oligonucleotide selected for hybridizing to the biomarker nucleic acid molecule, whether synthesized chemically or by recombinant DNA technology, is then isolated and purified using standard techniques and then preferably labeled (e.g., with ³⁵S or ³²P) using standard labeling protocols.

The present invention also contemplates the use of oligonucleotide pairs in polymerize chain reactions (PCR) to detect the expression of the biomarker in biological samples. The oligonucleotide pairs include a forward primer and a reverse primer.

The presence of biomarkers in a sample from a patient may be determined by nucleic acid hybridization, such as but not limited to Northern blot analysis, dot blotting, Southern blot analysis, fluorescence in situ hybridization (FISH), and PCR. Chromatography, preferably HPLC, and other known assays may also be used to determine messenger RNA levels of biomarkers in a sample.

Nucleic acid molecules encoding a biomarker of the present invention can be found in the biological fluids inside a biomarker-positive cancer cell that is being shed or released in a fluid or biological sample under investigation, e.g., urine. Nucleic acids encoding biomarkers can also be found directly (i.e., cell-free) in the fluid or biological sample.

In one aspect, the present invention contemplates the use of nucleic acids as agents for detecting biomarkers of the invention in biological samples of patients, wherein the nucleic acids are labeled. The nucleic agents may be labeled with a radioactive label, a fluorescent label, an enzyme, a chemiluminescent tag, a colorimetric tag or other labels or tags that are discussed above or that are known in the art.

In another aspect, the present invention contemplates the use of Northern blot analysis to detect the presence of biomarker mRNA in a sample of bodily fluid such as urine. The first step of the analysis involves separating a sample containing biomarker nucleic acid by gel electrophoresis. The dispersed nucleic acids are then transferred to a nitrocellulose filter or another filter. Subsequently, the labeled oligonucleotide is exposed to the filter under suitable hybridizing conditions, e.g., 50% formamide, 5×SSPE, 2×Denhardt's solution, 0.1% SDS at 42° C., as described in Molecular Cloning: A Laboratory Manual, Maniatis et al. (1982, CSH Laboratory). Other useful procedures known in the art include solution hybridization, dot and slot RNA hybridization, and probe based microarrays. Measuring the radioactivity of hybridized fragments, using standard procedures known in the art quantitates the amount of biomarker nucleic acid present in the biological fluid of a patient.

Dot blotting involves applying samples containing the nucleic acid of interest to a membrane. The nucleic acid can be denatured before or after application to the membrane. The membrane is incubated with a labeled probe. Dot blot procedures are well known to the skilled artisan and are described more fully in U.S. Pat. Nos. 4,582,789 and 4,617,261, the disclosures of which are incorporated herein by reference.

Polymerase chain reaction (PCR) is a process for amplifying one or more specific nucleic acid sequences present in a nucleic acid sample using primers and agents for polymerization and then detecting the amplified sequence. The extension product of one primer when hybridized to the other becomes a template for the production of the desired specific nucleic acid sequence, and vice versa, and the process is repeated as often as is necessary to produce the desired amount of the sequence. The skilled artisan to detect the presence of desired sequence (U.S. Pat. No. 4,683,195) routinely uses polymerase chain reaction.

A specific example of PCR that is routinely performed by the skilled artisan to detect desired sequences is reverse transcript PCR (RT-PCR; Saiki et al., Science, 1985, 230:1350; Scharf et al., Science, 1986, 233:1076). RT-PCR involves isolating total RNA from biological fluid, denaturing the RNA in the presence of primers that recognize the desired nucleic acid sequence, using the primers to generate a cDNA copy of the RNA by reverse transcription, amplifying the cDNA by PCR using specific primers, and detecting the amplified cDNA by electrophoresis or other methods known to the skilled artisan.

In a preferred embodiment, the methods of detecting biomarker nucleic acid in biological fluids of cancer patients or those at risk thereof, preferably urine of bladder cancer patients or those at risk thereof, include Northern blot analysis, dot blotting, Southern blot analysis, FISH, and PCR.

### Devices

The methods of the invention can be carried out on a solid support. The solid supports used may be those which are conventional for the purpose of assaying an analyte in a biological sample, and are typically constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. Biomarkers of the invention can be detected in a sample in vivo or in vitro (ex vivo). When, according to an embodiment of the invention, the amount of biomarker in a sample is to be determined without removing the sample from the body (i.e., in vivo, such as within the bladder), the support should be one which is harmless to the subject and may be in any form convenient for insertion into an appropriate part of the body. For example, the support may be a probe made of polytetrafluoroethylene, polystyrene or other rigid non-harmful plastic material and having a size and shape to enable it to be introduced into a subject. The selection of an appropriate inert support is within the competence of those skilled in the art, as are its dimensions for the intended purpose.

A contacting step in the assay (method) of the invention can involve contacting, combining, or mixing the biological sample and the solid support, such as a reaction vessel, microvessel, tube, microtube, well, multi-well plate, or other solid support. In an embodiment of the invention, the solid support to be contacted with the biological sample (e.g., urine) has an absorbent pad or membrane for lateral flow of the liquid medium to be assayed, such as those available from Millipore Corp. (Bedford, Mass.), including but not limited to Hi-Flow Plus™ membranes and membrane cards, and SureWick™ pad materials.

The diagnostic device useful in carrying out the methods of the invention can be constructed in any form adapted for the intended use. Thus, in one embodiment, the device of the invention can be constructed as a disposable or reusable test strip or stick to be contacted with a urine sample for which the presence of the biomarker or biomarker level is to be determined. In another embodiment, the device can be constructed using art recognized micro-scale manufacturing techniques to produce needle-like embodiments capable of being implanted or injected into an anatomical site, such as the bladder, for indwelling diagnostic applications. In other embodiments, devices intended for repeated laboratory use can be constructed in the form of an elongated probe or catheter, for sampling of urine, bladder wash, or bladder barbotage specimen.

In preferred embodiments, the devices of the invention comprise a solid support (such as a strip or dipstick), with a surface that functions as a lateral flow matrix defining a flow path for a biological sample such as urine.

Immunochromatographic assays, also known as lateral flow test strips or simply strip tests, for detecting various analytes of interest, have been known for some time, and may be used for detection of biomarkers of the invention. The benefits of lateral flow tests include a user-friendly format, rapid results, long-term stability over a wide range of climates, and relatively low cost to manufacture. These features make lateral flow tests ideal for applications involving home testing, rapid point of care testing, and testing in the field for various analytes. The principle behind the test is straightforward. Essentially, any ligand that can be bound to a visually detectable solid support, such as dyed microspheres, can be tested for, qualitatively, and in many cases even semi-quantitatively. For example, a one-step lateral flow immunostrip for the detection of free and total prostate specific antigen in serum is described in Fernandez-Sanchez et al. (J. Immuno. Methods, 2005, 307(1-2):1-12, which is incorporated herein by reference) and may be adapted for detection of biomarkers of the invention in a biological sample such as urine.

Some of the more common immunochromatographic assays currently on the market are tests for pregnancy (as an over-the-counter (OTC) test kit), Strep throat, and Chlamydia. Many new tests for well-known antigens have been recently developed using the immunochromatographic assay method. For instance, the antigen for the most common cause of community acquired pneumonia has been known since 1917, but a simple assay was developed only recently, and this was done using this simple test strip method (Murdoch, D. R. et al. J Clin Microbiol, 2001, 39:3495-3498). Human immunodeficiency virus (HIV) has been detected rapidly in pooled blood using a similar assay (Soroka, S. D. et al. J Clin Virol, 2003, 27:90-96). A nitrocellulose membrane card has also been used to diagnose schistosomiasis by detecting the movement and binding of nanoparticles of carbon (van Dam, G. J. et al. J Clin Microbiol, 2004, 42:5458-5461).

The two common approaches to the immunochromatographic assay are the non-competitive (or direct) and competitive (or competitive inhibition) reaction schemes (TechNote #303, Rev. 4001, 1999, Bangs Laboratories, Inc., Fishers, Ind.). The direct (double antibody sandwich) format is typically used when testing for larger analytes with multiple antigenic sites such as luteinizing hormone (LH), human chorionic gonadotropin (hCG), and HIV. In this instance, less than an excess of sample analyte is desired, so that some of the microspheres will not be captured at the capture line, and will continue to flow toward the second line of immobilized antibodies, the control zone. This control line uses species-specific anti-immunoglobulin antibodies, specific for the conjugate antibodies on the microspheres. Free antigen, if present, is introduced onto the device by adding sample (urine, serum, etc.) onto a sample addition pad. Free antigen then binds to antibody-microsphere complexes. Antibody 1, specific for epitope 1 of sample antigen, is coupled to dye microspheres and dried onto the device. When sample is added, microsphere-antibody complex is rehydrated and carried to a capture zone and control lines by liquid. Antibody 2, specific for a second antigenic site (epitope 2) of sample antigen, is dried onto a membrane at the capture line. Antibody 3, a species-specific, anti-immunoglobulin antibody that will react with antibody 1, is dried onto the membrane at the control line. If antigen is present in the sample (i.e., a positive test), it will bind by its two antigenic sites, to both antibody 1 (conjugated to microspheres) and antibody 2 (dried onto membrane at the capture line). Antibody 1-coated microspheres are bound by antibody 3 at the control line, whether antigen is present or not. If antigen is not present in the sample (a negative test), microspheres pass the capture line without being trapped, but are caught by the control line.

The competitive reaction scheme is typically used when testing for small molecules with single antigenic determinants, which cannot bond to two antibodies simultaneously. As with double antibody sandwich assay, free antigen, if present is introduced onto the device by adding sample onto a sample pad. Free antigen present in the sample binds to an antibody-microsphere complex. Antibody 1 is specific for sample antigen and couple to dyed microspheres. An antigen-carrier molecule (typically BSA) conjugate is dried onto a membrane at the capture line. Antibody 2 (Ab2) is dried onto the membrane at the control line, and is a species-specific anti-immunoglobulin that will capture the reagent particles and confirm that the test is complete. If antigen is present in the sample (a positive test), antibody on microspheres (Ab1) is already saturated with antigen from sample and, therefore, antigen conjugate bound at the capture line does not bind to it. Any microspheres not caught by the antigen carrier molecule can be caught by Ab2 on the control line. If antigen is not present in the sample (a negative test), antibody-coated dyed microspheres are allowed to be captured by antigen conjugate bound at the capture line.

Normally, the membranes used to hold the antibodies in place on these devices are made of primary hydrophobic materials, such as nitrocellulose. Both the microspheres used as the solid phase supports and the conjugate antibodies are hydrophobic, and their interaction with the membrane allows them to be effectively dried onto the membrane.

Samples and/or biomarker-specific binding agents may be arrayed on the solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or -1536 well, plates (or trays). Optionally, biomarker-specific binding agents may be immobilized on the solid support.

Detection of biomarkers of the invention and other cancer biomarkers, and other assays that are to be carried out on samples, can be carried out simultaneously or sequentially with the detection of other target molecules, and may be carried out in an automated fashion, in a high-throughput format.

The biomarker-specific binding agents can be deposited but “free” (non-immobilized) in the conjugate zone, and be immobilized in the capture zone of a solid support. The biomarker-specific binding agents may be immobilized by non-specific adsorption onto the support or by covalent bonding to the support, for example. Techniques for immobilizing binding agents on supports are known in the art and are described for example in U.S. Pat. Nos. 4,399,217, 4,381,291, 4,357,311, 4,343,312 and 4,260,678, which are incorporated herein by reference. Such techniques can be used to immobilize the binding agents in the invention. When the solid support is polytetrafluoroethylene, it is possible to couple hormone antibodies onto the support by activating the support using sodium and ammonia to aminate it and covalently bonding the antibody to the activated support by means of a carbodiimide reaction (yon Klitzing, Schultek, Strasburger, Fricke and Wood in “Radioimmunoassay and Related Procedures in Medicine 1982”, International Atomic Energy Agency. Vienna (1982), pages 57-62.).

The diagnostic device of the invention can utilize lateral flow strip (LFS) technology, which has been applied to a number of other rapid strip assay systems, such as over-the-counter early pregnancy test strips based on antibodies to human chorionic gonadotropin (hCG). As with many other diagnostic devices, the device utilizes a binding agent to bind the target molecule (biomarker of the invention). The device has an application zone for receiving a biological sample such as urine, a labeling zone containing label which binds to biomarker in the sample, and a detection zone where biomarker label is retained.

Binding agent retained in the detection zone gives a signal, and the signal differs depending on whether biomarker levels in the biological sample are lower than, equal to, or greater than a given threshold concentration. For example, in the case of a urinary biomarker of the invention for the detection of bladder cancer, the threshold concentration may be between 0 ng/ml or pg/ml and an upper limit of ng/ml or pg/ml. The methods used in the experiments described herein were capable of detecting as little as 50 pg (approximately 100 fmoles) of a 40 kDa protein, which is comparable to silver staining methods.

A sample from a subject having a biomarker level equal to or greater than the given reference biomarker concentration can be referred to as a “threshold level”, “threshold amount”, or “threshold sample”. The application zone in the device is suitable for receiving the biological sample to be assayed. It is typically formed from absorbent material such as blotting paper. The labeling zone contains binding agent that binds to any biomarker in the sample. In one embodiment, the binding agent is an antibody (e.g., monoclonal antibody, polyclonal antibody, antibody fragment). For ease of detection, the binding agent is preferably in association with a label that provides a signal that is visible to the naked eye, e.g., it is tagged with a fluorescent tag or a colored tag such as conjugated colloidal gold, which is visible as a pink color.

The detection zone retains biomarker to which the binding agent has bound. This will typically be achieved using an immobilized binding agent such as an immobilized antibody. Where the binding agent in the labeling zone and the detection zone are both antibodies, they will typically recognize different epitopes on the target molecule (biomarker protein). This allows the formation of a “sandwich” comprising antibody-biomarker-antibody.

The detection zone is downstream of the application zone, with the labelling zone typically located between the two. A sample will thus migrate from the application zone into the labeling zone, where any biomarker in the sample binds to the label. Biomarker-binding agent complexes continue to migrate into the detection zone together with excess binding agent. When the biomarker-binding agent complex encounters the capture reagent, the complex is retained whilst the sample and excess binding agent continue to migrate. As biomarker levels in the sample increase, the amount of binding agent (in the form of biomarker-binding agent complex) retained in the detection zone increases proportionally.

In preferred embodiments, the device of the invention has the ability to distinguish between samples according to the threshold concentration. This can be achieved in various ways.

One type of device includes a reference zone that includes a signal of fixed intensity against which the amount of binding agent retained in the detection zone can be compared—when the signal in the detection zone equals the signal in the reference zone, the sample is a threshold sample; when the signal in the detection zone is less intense than the reference zone, the sample contains less biomarker than a threshold sample; when the signal in the detection zone is more intense than the reference zone, the sample contains more biomarker than a threshold sample.

A suitable reference zone can be prepared and calibrated without difficulty. For this type of device, the binding agent will generally be present in excess to biomarker in the sample, and the reference zone may be upstream or, preferably, downstream of the detection zone. The signal in the reference zone will be of the same type as the signal in the detection zone, i.e., they will typically both be visible to the naked eye, e.g., they will use the same tag. A preferred reference zone in a device of this type comprises immobilized protein (e.g., bovine serum albumin) which is tagged with colloidal gold.

In another device of the invention, the reference zone is downstream of the detection zone and includes a reagent which captures binding agent (e.g., an immobilized anti-binding agent antibody). Binding agent that flows through the device is not present in excess, but is at a concentration such that 50% of it is bound by a sample having biomarker at the threshold concentration. In a threshold sample, therefore, 50% of the binding agent will be retained in the detection zone and 50% in the reference zone. If the biomarker level in the sample is greater than in a threshold sample, less than 50% of the binding agent will reach the reference zone and the detection zone will give a more intense signal than the reference zone; conversely, if the biomarker level in the sample is less than in a threshold sample, less than 50% of the binding agent will be retained in the detection zone and the reference zone will give a more intense signal than the detection zone.

In another device of the invention which operates according to similar principles, the reference zone is downstream of the detection zone and includes a limiting amount of a reagent which captures binding agent (e.g., an immobilized anti-binding agent antibody). The reagent is present at a level such that it retains the same amount of label which would bind to the detection zone for a threshold sample, with excess label continuing to migrate beyond the reference zone.

In these three types of device, therefore, a comparison between the detection zone and the reference zone is used to compare the sample with the threshold concentration. The detection:reference binding ratio can preferably be determined by eye. Close juxtaposition of the detection and reference zones is preferred in order to facilitate visual comparison of the signal intensities in the two zones.

In a fourth type of device, no reference zone is needed, but the detection zone is configured such that it gives an essentially on/off response, e.g., no signal is given below the threshold concentration but, at or above the threshold, signal is given.

In a fifth type of device, no reference zone is needed, but an external reference is used which corresponds to the threshold concentration. This can take various forms, e.g., a printed card against which the signal in the detection zone can be compared, or a machine reader which compares an absolute value measured in the detection zone (e.g., a calorimetric signal) against a reference value stored in the machine.

In some embodiments of the invention, the device includes a control zone downstream of the detection zone. This will generally be used to capture excess binding agent that passes through the detection and/or reference zones (e.g., using immobilized anti-binding agent antibody). When binding agent is retained at the control zone, this confirms that mobilization of the binding agent and migration through the device have both occurred. It will be appreciated that this function may be achieved by the reference zone.

In a preferred embodiment, the detection, reference and control zones are preferably formed on a nitrocellulose support.

Migration from the application zone to the detection zone will generally be assisted by a wick downstream of the detection zone to aid capillary movement. This wick is typically formed from absorbent material such as blotting or chromatography paper.

The device of the invention can be produced simply and cheaply, conveniently in the form of a dipstick. Furthermore, it can be used very easily, for instance by the home user. The invention thus provides a device which can be used at home as a screen for bladder cancer.

### Kits for Diagnosing or Monitoring a Urogenital Cancer

The present invention also concerns kits comprising the required elements for diagnosing or monitoring cancer, including bladder cancer. In one embodiment, the kits comprise one or more containers for collecting a biological fluid or sample from a patient and an agent for detecting the presence of biomarker polypeptides or nucleic acids encoding the polypeptides in the fluid. The biomarker components of the kits can be packaged either in aqueous medium or in lyophilized form.

The methods of the invention can be carried out using a diagnostic kit for qualitatively or quantitatively detecting one or more biomarkers of the invention in a sample such as urine. By way of example, the kit can contain binding agents (e.g., antibodies) specific for biomarkers of the invention, antibodies labeled with a detectable label or substance that can bind to the binding agents. If the detectable substance is an enzyme, then the kit can comprise a substrate for the enzyme. The kit can also contain a solid support (such as microtiter multi-well plates, nitrocellulose, etc.) standards, assay diluent, wash buffer, adhesive plate covers, and/or instructions for carrying out a method of the invention using the kit. In one embodiment, the kit includes one or more protease inhibitors (e.g., a protease inhibitor cocktail) and/or nuclease inhibitors to be applied to the biological sample to be assayed (such as blood or urine).

Kits for diagnosing or monitoring cancer containing one or more agents that detect the biomarker polypeptides, such as but not limited to biomarker antibodies, fragments thereof, or biomarker binding partners, can be prepared. The agent(s) can be packaged with a container for collecting the biological sample from a patient. When the antibodies or binding partner are used in the kits in the form of conjugates in which a label is attached, such as a radioactive metal ion or a moiety, the components of such conjugates can be supplied either in fully conjugated form, in the form of intermediates, or as separate moieties to be conjugated by the user of the kit.

Kits containing one or more agents that detect biomarker nucleic acids, such as but not limited to the full length biomarker nucleic acid, biomarker oligonucleotides, and pairs of biomarker primers can also be prepared. The agent(s) can be packaged with a container for collecting biological samples from a patient. The nucleic acid can be in the labeled form or unlabeled form.

Other components of the kit may include, but are not limited to, means for collecting biological samples, means for labeling the detecting agent (binding agent), means for immobilizing the biomarker protein or biomarker nucleic acid in the biological sample (e.g., support membranes, nitrocellulose, etc.), means for applying the biological sample to an immobilizing support means for binding the agent to the biomarker in the biological sample of a subject, a second antibody, a means for isolating total RNA or mRNA from a biological fluid of a subject, means for performing gel electrophoresis, means for generating cDNA from isolated total RNA or mRNA, means for performing hybridization assays, and means for performing PCR, etc.

As used herein, the term “ELISA” includes an enzyme-linked immunoabsorbent assay that employs an antibody or antigen bound to a solid phase and an enzyme-antigen or enzyme-antibody conjugate to detect and quantify the amount of an antigen (e.g., biomarker of the invention) or antibody present in a sample. A description of the ELISA technique is found in Chapter 22 of the 4^{th} Edition of Basic and Clinical Immunology by D. P. Sites et al., 1982, published by Lange Medical Publications of Los Altos, Calif. and in U.S. Pat. Nos. 3,654,090; 3,850,752; and 4,016,043, the disclosures of which are herein incorporated by reference. ELISA is an assay that can be used to quantitate the amount of antigen, proteins, or other molecules of interest in a sample. In particular, ELISA can be carried out by attaching on a solid support (e.g., polyvinylchloride) an antibody specific for an antigen or protein of interest. Cell extract or other biological sample of interest such as urine can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

In these exemplary embodiments, the antibodies can be labeled with pairs of FRET dyes, bioluminescence resonance energy transfer (BRET) protein, fluorescent dye-quencher dye combinations, beta gal complementation assays protein fragments. The antibodies may participate in FRET, BRET, fluorescence quenching or beta-gal complementation to generate fluorescence, colorimetric or enhanced chemiluminescence (ECL) signals, for example.

These methods are routinely employed in the detection of antigen-specific antibody responses, and are well described in general immunology text books such as Immunology by Ivan Roitt, Jonathan Brostoff and David Male (London: Mosby, c1998. 5th ed. and Immunobiology: Immune System in Health and Disease/Charles A. Janeway and Paul Travers. Oxford: Blackwell Sci. Pub., 1994), the contents of which are herein incorporated by reference.

### DEFINITIONS

As used herein, the terms “cancer” and “cancerous” refer to or describe the physiological condition in mammals that is typically characterized by unregulated cell growth, i.e., proliferative disorders. Examples of such proliferative disorders include cancers such as carcinoma, lymphoma, blastoma, sarcoma, and leukemia, as well as other cancers disclosed herein. More particular examples of such cancers include bladder cancer, breast cancer, prostate cancer, colon cancer, squamous cell cancer, small-cell lung cancer, non-small cell lung cancer, gastrointestinal cancer, pancreatic cancer, cervical cancer, ovarian cancer, peritoneal cancer, liver cancer, e.g., hepatic carcinoma, colorectal cancer, endometrial carcinoma, kidney cancer, and thyroid cancer.

As used herein, the term “bladder cancer” includes transitional cell (urothelial) carcinoma, squamous cell carcinoma, and adenocarcinoma that has spread to the bladder. Risk factors for bladder cancer include, but are not limited to, cigarette smoking, occupational exposure (such as workers in the rubber, chemical, leather, textile, metal, and printing industries that are exposed to substances such as anihiline dye and aromatic amines; hairdressers, machinists, painters, and truck drivers), chronic bladder irritation (e.g., bladder infection or bladder stones), being of increased age, being of male gender, being of Caucasian race, having a personal history of bladder cancer, having a family history of bladder cancer, and certain parasitic infections.

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), ultrasound, CT, and MRI, or by needle aspirations. The use of these latter techniques is more common in early detection. Molecular and phenotypic analysis of cancer cells within a tissue will usually confirm if the cancer is endogenous to the tissue or if the lesion is due to metastasis from another site.

A “sample” (biological sample) can be any composition of matter of interest from a human or non-human subject, in any physical state (e.g., solid, liquid, semi-solid, vapor) and of any complexity. The sample can be any composition reasonably suspecting of containing one or more biomarkers of the invention that can be analyzed by the methods, devices, and kits of the invention. Preferably, the sample is a fluid (biological fluid such as urine). Samples can include human or animal samples. The sample may be contained within a test tube, culture vessel, multi-well plate, or any other container or supporting substrate. The sample can be, for example, a cell culture, human or animal tissue. Fluid homogenates of cellular tissues are biological fluids that may contain biomarkers for detection by the invention.

The “complexity” of a sample refers to the relative number of different molecular species that are present in the sample.

The terms “body fluid” and “bodily fluid”, as used herein, refer to a composition obtained from a human or animal subject. Bodily fluids include, but are not limited to, urine, bladder wash, bladder barbotage specimen, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningal fluid, amniotic fluid, glandular fluid, feces, perspiration, mucous, vaginal or urethral secretion, cerebrospinal fluid, and transdermal exudate. Bodily fluid also includes experimentally separated fractions of all of the preceding solutions or mixtures containing homogenized solid material, such as feces, tissues, and biopsy samples.

The term “ex vivo,” as used herein, refers to an environment outside of a subject. Accordingly, a sample of bodily fluid collected from a subject is an ex vivo sample of bodily fluid as contemplated by the subject invention. In-dwelling embodiments of the method and device of the invention obtain samples in vivo.

As used herein, the term “conjugate” refers to a compound comprising two or more molecules bound together, optionally through a linking group, to form a single structure. The binding can be made by a direct connection (e.g., a chemical bond) between the molecules or by use of a linking group.

As used herein, the terms solid “support”, “substrate”, and “surface” refer to a solid phase which is a porous or non-porous water insoluble material that can have any of a number of shapes, such as strip, rod, particle, beads, or multi-welled plate. In some embodiments, the support has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified Teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, test strips, tubing, spheres, containers, capillaries, pads, slices, films, plates, slides, etc., depending upon the particular application. Preferably, the solid support is planar in shape, to facilitate contact with a biological sample such as urine, whole blood, plasma, serum, peritoneal fluid, or ascites fluid. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., Hi-Flow™ Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating).

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group.

As used herein, the term “receptor” and “receptor protein” are used herein to indicate a biologically active proteinaceous molecule that specifically binds to (or with) other molecules such as biomarkers of the invention.

As used herein, the term “ligand” refers to a molecule that contains a structural portion that is bound by specific interaction with a particular receptor protein.

As used herein, the term “antibody” refers to immunoglobulin molecules and immunologically active portions (fragments) of immunoglobulin molecules, i.e., molecules that contain an antibody combining site or paratope. The term is inclusive of monoclonal antibodies and polyclonal antibodies.

As used here, the terms “monoclonal antibody” or “monoclonal antibody composition” refer to an antibody molecule that contains only one species of antibody combining site capable of immunoreacting with a particular antigen. A monoclonal antibody composition thus typically displays a single binding affinity for any antigen with which it immunoreacts. A monoclonal antibody composition is typically composed of antibodies produced by clones of a single cell called a hybridoma that secretes (produces) only one type of antibody molecule. The hybridoma cell is formed by fusing an antibody-producing cell and a myeloma or other self-perpetuating cell line. Such antibodies were first described by Kohler and Milstein, Nature, 1975, 256:495-497, the disclosure of which is herein incorporated by reference. An exemplary hybridoma technology is described by Niman et al., Proc. Natl. Acad. Sci. U.S.A., 1983, 80:4949-4953. Other methods of producing monoclonal antibodies, a hybridoma cell, or a hybridoma cell culture are also well known. See e.g., Antibodies: A Laboratory Manual, Harlow et al., Cold Spring Harbor Laboratory, 1988; or the method of isolating monoclonal antibodies from an immunological repertoise as described by Sasatry, et al., Proc. Natl. Acad. Sci. USA, 1989, 86:5728-5732; and Huse et al., Science, 1981, 246:1275-1281. The references cited are hereby incorporated herein by reference.

As used herein, a semi-permeable membrane refers to a bio-compatible material which is impermeable to liquids and capable of allowing the transfer of gases through it. Such gases include, but are not limited to, oxygen, water vapor, and carbon dioxide. Semi-permeable membranes are an example of a material that can be used to form a least a portion of an enclosure defining a flow chamber cavity. The semi-permeable membrane may be capable of excluding microbial contamination (e.g., the pore size is characteristically small enough to exclude the passage of microbes that can contaminate the analyte, such as cells). In a particular aspect, a semi-permeable membrane can have an optical transparency and clarity sufficient for permitting observation of an analyte, such as cells, for color, growth, size, morphology, imaging, and other purposes well known in the art.

As used herein, the term “bind” refers to any physical attachment or close association, which may be permanent or temporary. The binding can result from hydrogen bonding, hydrophobic forces, van der Waals forces, covalent, or ionic bonding, for example.

As used herein, the term “particle” includes insoluble materials of any configuration, including, but not limited to, spherical, thread-like, brush-like, and irregular shapes. Particles can be porous with regular or random channels inside. Particles can be magnetic. Examples of particles include, but are not limited to, silica, cellulose, Sepharose beads, polystyrene (solid, porous, derivatized) beads, controlled-pore glass, gel beads, magnetic beads, sols, biological cells, subcellular particles, microorganisms (protozoans, bacteria, yeast, viruses, and other infectious agents), micelles, liposomes, cyclodextrins, and other insoluble materials.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences.

As used herein, the term “polypeptide” refers to any polymer comprising any number of two or more amino acids, and is used interchangeably herein with the terms “protein”, “gene product”, and “peptide”.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates.

The terms “polynucleotide”, “nucleic acid molecule”, and “nucleotide molecule” are used interchangeably herein and refer to a polymer of nucleotides joined together by a phosphodiester linkage between 5′ and 3′ carbon atoms. Polynucleotides can encode a polypeptide such as biomarker polypeptide (whether expressed or non-expressed), or may be short interfering RNA (siRNA), antisense nucleic acids (antisense oligonucleotides), aptamers, ribozymes (catalytic RNA), or triplex-forming oligonucleotides (i.e., antigene), for example.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog” or “nucleic acid analog”, also referred to herein as an altered nucleotide/nucleic acid or modified nucleotide/nucleic acid refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. For example, locked nucleic acids (LNA) are a class of nucleotide analogs possessing very high affinity and excellent specificity toward complementary DNA and RNA. LNA oligonucleotides have been applied as antisense molecules both in vitro and in vivo (Jepsen J. S. et al., Oligonucleotides, 2004, 14(2):130-146).

As used herein, the term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA).

The terms “comprising”, “consisting of” and “consisting essentially of” are defined according to their standard meaning. The terms may be substituted for one another throughout the instant application in order to attach the specific meaning associated with each term.

The terms “isolated” or “biologically pure” refer to material that is substantially or essentially free from components which normally accompany the material as it is found in its native state.

As used in this specification, the singular forms “a”, “an”, and “the” include plural reference unless the context clearly dictates otherwise. Thus, for example, a reference to “a biomarker” includes more than one such biomarker. A reference to “an antibody” includes more than one such antibody. A reference to “a molecule” includes more than one such molecule, and so forth.

The practice of the present invention can employ, unless otherwise indicated, conventional techniques of molecular biology, microbiology, recombinant DNA technology, electrophysiology, and pharmacology that are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods hi Enzymology (S. Colowick and N. Kaplan Eds., Academic Press, Inc.); Transcription and Translation (Hames et al. Eds. 1984); Gene Transfer Vectors For Mammalian Cells (J. H. Miller et al. Eds. (1987) Cold Spring Harbor Laboratory, Cold. Spring Harbor, N.Y.); Scopes, Protein Purification: Principles and Practice (2nd ed., Springer-Verlag); and PCR: A Practical Approach (McPherson et al. Eds. (1991) IRL Press)), each of which are incorporated herein by reference in their entirety.

Following are examples that illustrate materials, methods, and procedures for practicing the invention. The examples are illustrative and should not be construed as limiting.

### Materials and Methods

Urine collection. Urine specimens were collected from University of Florida urology clinic at Shands & UF Hospital, Jacksonville, Fla. Eight samples of urine were collected from patients cystoscopically confirmed to have bladder cancer (n=5) or to have non-tumor conditions (n=3). An additional two normal donor urine specimens (n=2) were obtained from asymptomatic volunteers. Samples were collected from patients visiting the clinic according to University of Florida IRB approved consent protocols. Each sample consisted of 15-50 mL of mid-stream urine collected in a sterile cup. All specimens were processed in the same way, with no special treatment being performed to remove occasional blood prior to analysis in order to preserve the intrinsic components. Table 1 shows patient clinical details.

TABLE 1
Clinicopathological data of study samples.

| Patient Sample | Disease Status | Sex | Age |
|---|---|---|---|
| Tumor-bearing | | | |
| T4 | low-grade bladder cancer | female | 53 |
| T3 | high-grade bladder cancer | male | 52 |
| T9 | high-grade bladder cancer | male | 71 |
| T8 | high-grade bladder cancer | female | 75 |
| T13 | high-grade bladder cancer | male | 79 |
| Non tumor-bearing | | | |
| N3 | hematuria, no tumor | male | 66 |
| N10 | hematuria, no tumor | male | ND |
| N2 | hematuria, no tumor | male | 53 |
| N5 | asymptomatic donor | female | 28 |
| N7 | asymptomatic donor | female | 29 |

Sample preparation. Cells and debris were removed from urine samples by centrifugation at 5000×g for 10 min at RT, and supernatant was stored frozen at −80° C. until analysis. Four times the sample volumes of cold (−20° C.) acetone was added to the urine specimen for protein precipitation. The sample was left at 20° C. for 1 hour followed by centrifugation at 12 000×g for 15 min at 4° C. The supernatant was removed. The tube was left open at room temperature to partially remove the remaining solvent. The pellet was resuspended in binding buffer (20 mM Tris, 0.15 M NaCl, 1 mM MnCl₂ and 1 mM CaCl₂, pH 7.4). The sample was vortexed vigorously to completely dissolve the pelleted protein. The protein amounts in sample concentrates were determined using the Bradford protein assay (Bio-Rad, Hercules, Calif.).

Con A lectin affinity chromatography. Agarose bound lectins, Agarose bound Concanavalin A (Con A), was purchased from Vector Laboratories (Burlingame, Calif., USA). 500 μL agarose bound Con A was packed into a disposable screw end-cap spin column with filters at both ends. The column was first washed with 500 μL binding buffer (20 mM Tris, 1 mM MnCl₂, 1 mM CaCl₂, 0.15 M NaCl, pH 7.4) three times by centrifuging the spin column at 500 rpm for 2 min. Urine sample diluted with 500-800 μL binding buffer was loaded onto the column and incubated for 15 min. The column was centrifuged for 2 mM at 500 rpm to remove the non-binding fraction. The column was washed with 600 μL binding buffer twice to wash off the non-specific binding. The captured glycoproteins were released with 250 μL elution buffer (0.4M methyl-α-D-mannopyroside in 20 mM Tris and 0.5 M NaCl, pH 7.0) and the eluted fraction was collected by centrifugation at 500 rpm for 2 min. This step was repeated twice and the eluted fractions were pooled. Total protein concentration of fractions was measured using standard methods (e.g., Bradford assay).

Tryptic digestion/PNGase F treatment. The sample was concentrated using Microcon YM-10 (Millipore Corp., Bradford, Mass.) according to the manufacture protocol. Approximately 10 μL of sample was collected from filter device. 40 μL of 50 mM ammonium bicarbonate buffer (Sigma) at pH 7.8 was added to the sample. The reduction was done by adding 5 μL of 10 mM DTT (Sigma) and the sample was incubated at 45° C. for 30 min. The reduction reaction was terminated by incubating the sample at 75° C. for 10 min. 1 μg of modified sequencing grade trypsin (Promega, Madison, Wis.) was added for digestion. The samples were vortexed again and incubated at 37° C. for 18 hours. The digestion was terminated by adding 1 μL TFA to the digest. Tryptic digestion mixture was completely dried using a SpeedVac concentrator (Labconco Corp., Kansas City, Mo.) operated at 45° C. The digest was reconstituted with 20 μL HPLC grade water (Fisher Scientific, Hanover Park, Ill.) and half of the volume of the sample (10 μL) was kept at −20° C. for later analysis by LC/MS/MS. 50 μL of 100 mM ammonium bicarbonate and 1 unit of PNGase F (Sigma) were added to the rest of the digest. The deglycosylation reaction was incubated for 24 h in a water bath set at 37° C. The reaction was stopped by incubating the digest mixture at 75° C. for 20 min. The sample was dried using a SpeedVac and kept at −20° C. for LC/MS/MS analysis.

LC/MS/MS. A Paradigm MG4 micropump (Michrom Biosciences Inc., Auburn, Calif.) was used for chromatographic separation of peptide mixtures. A mobile phase system of two solvents was used, where solvent A and B were composed of 0.1% formic acid (Sigma) in HPLC grade water and ACN (Sigma), respectively. Prior to LC/MS/MS, the dried tryptic digest was reconstituted in HPLC grade water. The final concentration of tryptic digest mixture of ˜0.25-1 ng/μL was injected through a 20 μL fixed sample loop. The peptide mixture was loaded into a desalting column packed with C4: 300 μm i.d.×50 mm (Michrom Biosciences Inc.) and washed with 95% solvent A and 5% solvent B with a flow rate of 0.3 μL/min for 5 minutes. The peptides were eluted onto an analytical C18 column (100 μm i.d.×150 mm) (Michrom Biosciences Inc.) over 45 min using a step gradient with a flow rate of 300 nL/min. The 40-min gradient was 5%-45% B in 25 min, 45-95% B in 10 min and held at 95% B for 5 min. The resolved peptides were analyzed on a linear ion trap MS with a nano-LC ESI source (LTQ, Thermo Finnigan, San Jose, Calif.). The capillary transfer tube was set at 200° C. and ESI spray voltage at 2.5 kV and capillary voltage at 30 V. The ion activation was achieved by utilizing helium at normalized collision energy of 35%. The data acquisition and generation of peak list files was automatically done by Xcaliber software. For each cycle of one full mass scan (range of m/z 400-2000), the three most intense ions in the spectrum were selected for tandem MS analysis, unless they appeared in the dynamic or mass exclusion lists. Precursor selection was based upon a nomialized threshold of 30 counts/s. Multiple analyses (up to 10 LC/MS/MS runs) were performed for each sample. Dynamic exclusion was employed in the LC/MS/MS analysis in order to increase the number of identified proteins. In few cases, precursor ion mass of high abundant proteins such as albumin or uromodulin were listed for mass exclusion when their mass hindered the detection of relatively low abundant proteins.

Database searching and manual validation. All MS/MS spectra were analyzed by TurboSequest of Bioworks software version 3.1 SR1 (Thermo Finnigan). Peptide fragment lists were generated and submitted to Swiss-Prot database searching. The search parameters were as follows; 1) database species: Homo Sapiens; 2) allowing two missed cleavage; 3) possible modifications: oxidation of M; 4) peptide ion mass tolerance 1.50 Da; 5) fragment ion mass tolerance 0.0 Da; and 6) peptide charge +1, +2, +3. The filter function in Bioworks browser was used to set a single threshold to consider fully tryptic peptides assigned with Xcorr (Chen, E. I. et al. Mol Cell Proteomics, 2006, 5(1):53-6) values of the following; ≧1.5 for singly charged ions, ≧2.5 for doubly charged ions, and ≧3.5 for triply charged ions, while no ions at higher charged states were considered. After that, the search results that passed the first criteria were subjected to manual inspection, in which ΔCn≧0.1 was considered. Data reduction was then manually performed using the stringent criteria as follows; 1) Sp>500, for >6 residual peptides; 200<Sp<500, for <6 residual peptides, 2) Rsp<5, 3) Ions>70%, 4) less than 2 potential modification sites were considered, 5) If the same spectrum matched different proteins, the higher Rsp, higher ΔCn and/or ions>70% would be selected. In other words, peptide ion scans must correspond to a single accepted protein. All SEQUEST search parameters and data filtering were the same in all digest fractions except the modification of asparagine was allowed by +1 Da in the tryptic digest/PNGase F fractions. The +1 Da was allowed owing to hydrolysis of amide of the asparagine side chain to release the asparagine-linked oligosaccharides from glycopeptides.

Protein ids were accepted if, and only if, the id was positively identified in at least 2 MS/MS analyses. If the protein were identified by a single peptide matching, the spectrum was manually validated. The matched ions covering at least 70% of the peptide sequence must have high signal-to-noise ratio. Two high s/n spectra of a single peptide matching protein were accepted for positive identification.

It should be understood that the examples and embodiments described herein are for illustrative purposes only and that various modifications or changes in light thereof will be suggested to persons skilled in the art and are to be included within the spirit and purview of this application.

### Example 1

### Urinary Protein Content

In the present study, the glycoprotein profile of urine obtained from patients was investigated and compared with cystoscopically confirmed bladder cancer or no evidence of bladder cancer (Table 1). Protein concentration of normal urine is estimated to be less than 100 mg/L (Adachi, J. et aL Genome Biol, 2006, 7(9):R80). The measurements of urinary protein concentrations from non tumor-bearing patients in the present study were consistent with the reported value (average of 31 mg/L) except for one patient that showed a sign of proteinuria, (defined as >150 mg/day protein) most likely due to a high level of hematuria. The average protein concentration urine from tumor-bearing patients was 127 mg/L. The greater amount of protein recovered from bladder cancer patients' urine meant that 7-15 mL of urine was typically sufficient for this analysis; however, a larger volume of asymptomatic patient urine was required, i.e., 15-30 mL. Agarose-bound Concanavalin A (Con A) was chosen to isolate glycoproteins in human urine owing to its broad specificity, preferential binding to oligomannosidic, hybrid and bi-antennary N-glycans (Wuhrer, M. et al. “Glycoproteomics based on tandem mass spectrometry of glycopeptides” J Chromatogr B Analyt Technol Biomed Life Sci, Oct. 16, 2006, Epub ahead of print). Con A-capture experiments were performed in duplicate with starting material of lower than 400 μg. The measured protein content of eluted fractions from urine samples indicated that ˜4% of total input protein bound to the lectin column.

### Example 2

### Mass Spectrometric Analysis

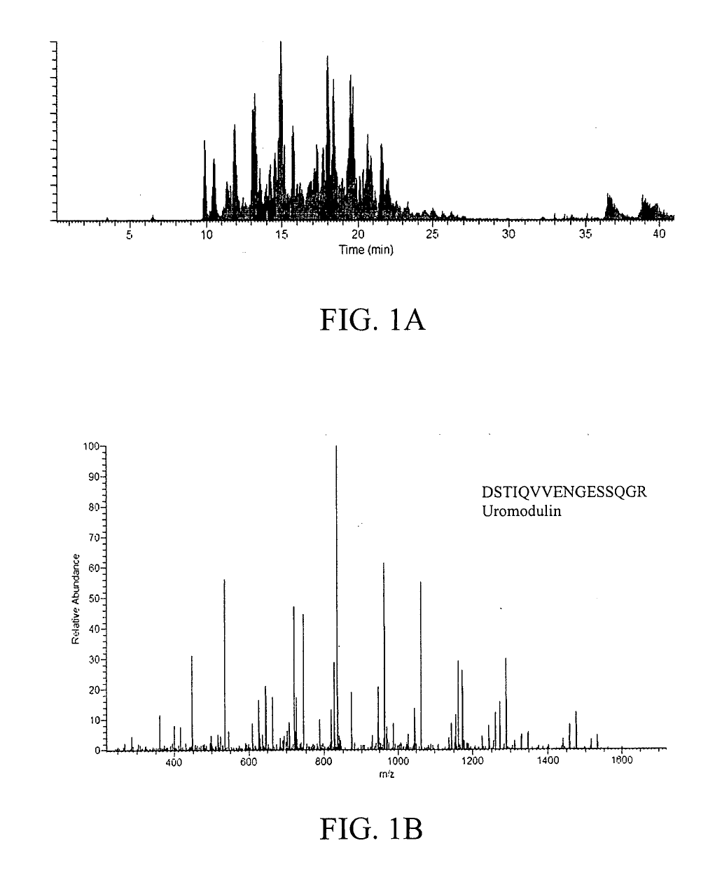

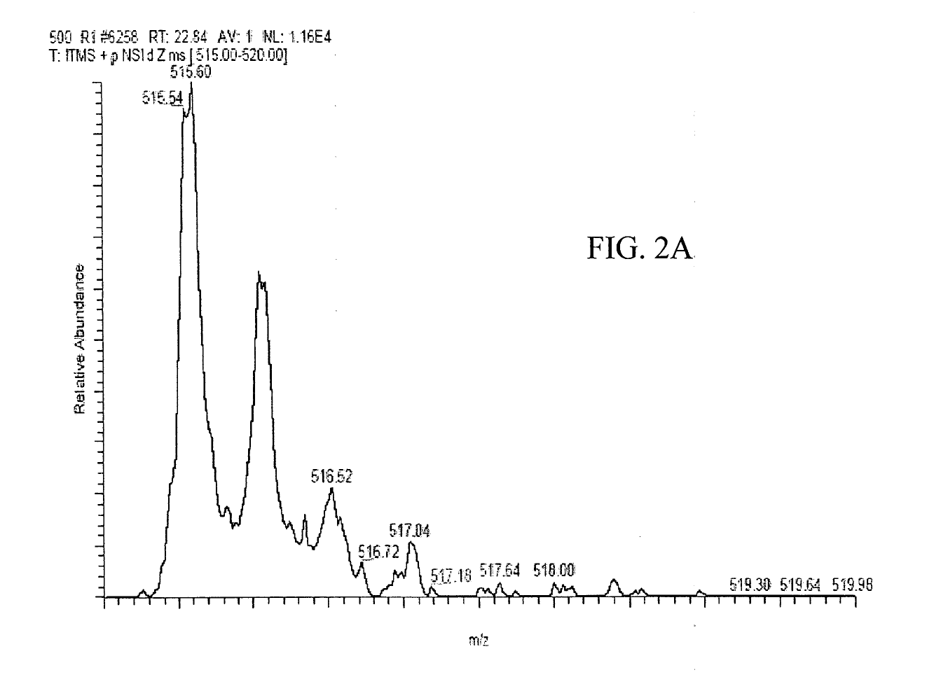

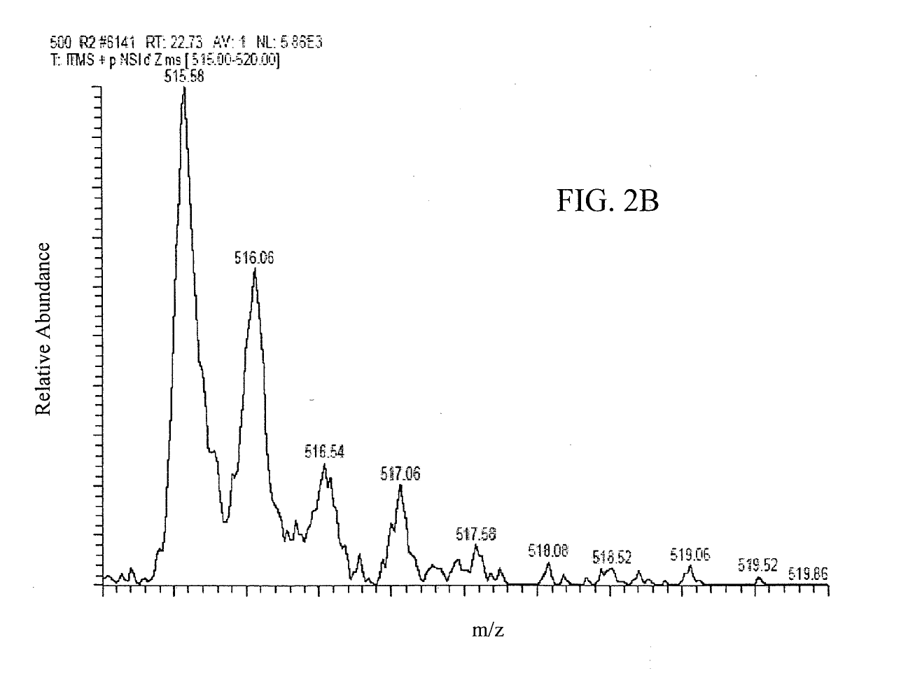

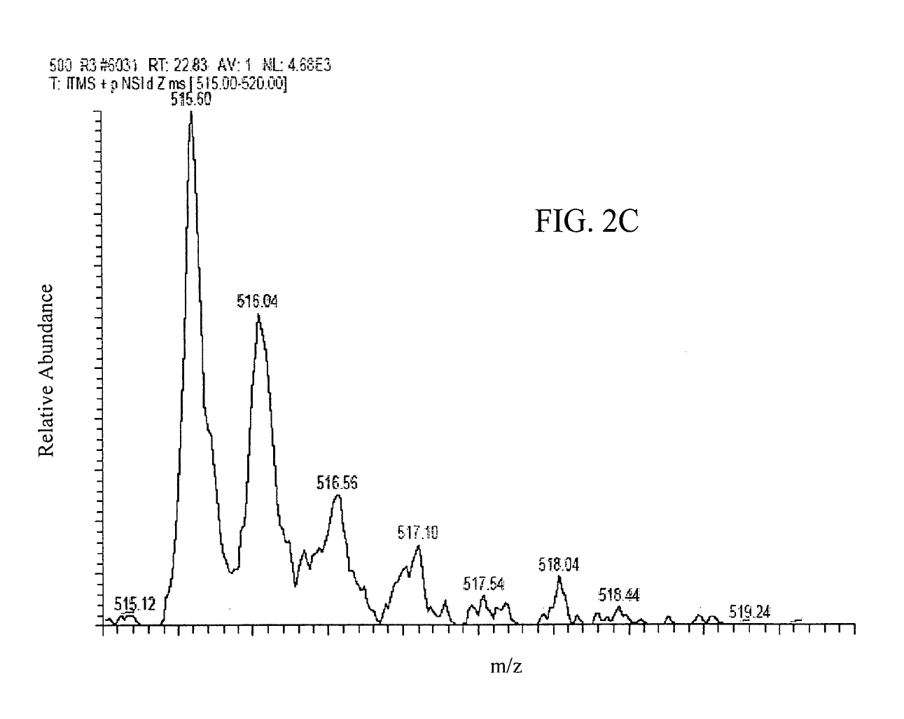

Con A bound proteins were subjected to mass spectrometric analysis. A semi-shotgun approach was used where the bound fractions were enzymatically digested and analyzed by nano-LC/MS/MS. For each scan, the three most abundant peptides were sequenced. FIG. 1A is a representative nano-LC/MS/MS base peak chromatogram, showing the detection of the more abundant ions across a 40-min gradient separation. The peak capacity of the nano-LC separation was estimated to be ˜60 to 120 based on examining the full-width-at-half-maximum (fwhm). This implies that >60 peptides can be sequenced within a single run; however, it is important to note that multiple peptides were detected within a single resolved peak from all experimentally based peak chromatograms, which is commonly observed from LC/MS/MS analysis of highly complex samples. FIG. 1B shows a representative MS/MS spectrum of a peptide sequence from uromodulin, one of the most abundant proteins in urine, identified in the eluted fraction of a cancer-bearing patient sample. Eight other ion peaks was also detected and assigned to this protein. Uromodulin is an N-linked glycoprotein as annotated by Swiss-Prot and was detected in 7 of 10 urine samples. The reproducibility of the Con A-captured experiment coupled to nano-LC/MS/MS was evaluated by running samples in triplicate in independently packed Con-A affinity columns. The data showed that the Con A-captured experiments were very reproducible. Approximately 75% of identified proteins were N-linked glycoproteins and these were observed in all three parallel, lectin-captured samples (Table 2). To evaluate reproducibility of nano LC-MS/MS analysis alone, the retention time of several peptides detected in a normal captured fraction was performed. FIGS. 2A-2C show representative MS spectra of the +2 precursor mass of YFIDFVAR from the protein kinnogen-1 in three independent runs. The retention times of this peptide in these runs were not significantly different. Based on the results, the standard deviation of the elution time of a peptide was estimated to be less than 10 seconds in the experimental system employing a capillary C18 column.

TABLE 2
Reproducibility study of Con A lectin-captured urinary proteins.
Proteins from a tumor-bearing patient urine sample were loaded
equally onto three replicate lectin affinity columns. The number
of proteins identified in each eluted fraction is summarized.

| | | N-linked |
|---|---|---|
| Con A-captured sample | Protein ID | glycoproteins^{a} |
| 1 | 35 | 25 |
| 2 | 31 | 25 |
| 3 | 35 | 27 |
| Common proteins^{b} | 25 | 20 |

^{a}N-linked Glycoproteins as annotated in Swiss-Prot database and as predicted N-linked glycoproteins by the software NetNGlyc 1.0 server.
^{b}A number of proteins found in all three Con A-captured samples.

### Example 3

### Glycoprotein Identification

Both tryptic digest fractions and tryptic digest/PNGase F fractions were analyzed and their results were combined to increase confidence in protein identification. Removing glycans from digested peptides with PNGase F is reported to provide a stronger signal for the peptide ions compared to the glycan remaining intact (Wang, Y. et al. Glycobiology, 2006, 16(6):514-23). Based on the current results, it was indeed found that glycopeptides were poorly identified when only the tryptic digest fractions were analyzed, but after tryptic digest/PNGase F treatment, a number of glycopeptides were positively identified from the same fraction. In the experiments of this study, all SEQUEST search parameters and data filtering were the same, except that 1 Da was allowed for modification of Asn for deglycosylated digests. Table 3 lists the peptides that were not detected in the fraction with the tryptic digestion alone, where # denotes the glycosylated site of the peptide. These peptides have consensus sequence NXS/T, where X is not proline, and these sites have been annotated using the Swiss-Prot database. Several potential novel glycopeptides were identified using this approach. Locating glycosylation sites was not the primary goal of the experiments, but the data supports the claims of others that improved signals for glycopeptides are revealed when their glycans have been enzymatically released (Wang, Y. et al. Glycobiology, 2006, 16(6):514-23). The analysis of the deglycosylated fraction as well as the tryptic digest fraction facilitated detection of unique peptide sequences and increased the total number of positive protein ids. Accurate identification of components in a complex biological material, such as urinary proteomes, using LC tandem mass spectrometry technique, requires strict attention to data interpretation and handling. Multiple analyses of samples and manual data validation with stringent criteria are particularly important for correct identification. In this study, the Xcorr for each ion charge and ΔCn were set at the accepted values for high confidence of protein identification (Chen, E. I. et al. Mol Cell Proteomics, 2006, 5(1):53-6). In addition, Rsp, Sp, and % ion match were also considered in order to increase the confidence level. Visual assessment of spectra was conducted if a single peptide resulted in a positive protein id and was detected in at least two LC/MS/MS runs of the same sample. The protein was considered present in the sample if its corresponding peptides passed all the stringent criteria (described in Materials and Methods), and the protein was observed in at least 2 MS/MS analyses.

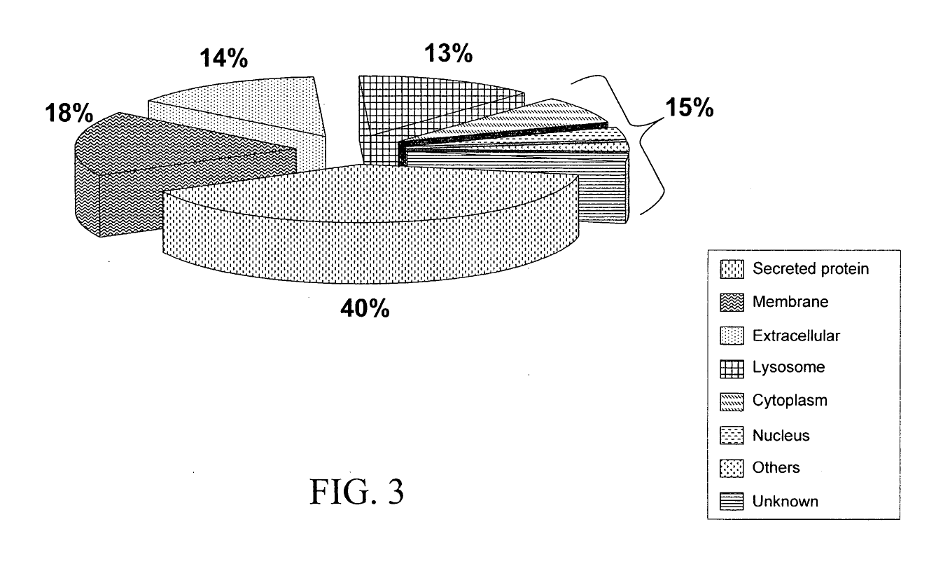

A total of 186 urinary proteins were positively identified in this study, with ˜40-65 proteins being detected in each urine specimen. Table 5 summarizes all of the protein IDs obtained from analysis of all of the urine specimens. The majority of the glycoproteins had molecular masses within the range 30,000 to 80,000 kDa, but glycoproteins as large as 300,000 kDa were detected. Of the 186 identified proteins, 128 (69%) were glycoproteins as annotated by the Swiss-Prot database. Other proteins could be accounted for through non-specific lectin column binding, or unconventional glycan sites. Not surprisingly, FIG. 3 shows that the majority of identified proteins were secreted (40%), membrane proteins (18%), and extracellular proteins (14%). Zinc-alpha-2-glycoprotein and alpha-1-microglobulin (AMBP protein) were the most frequent positive protein ids and were identified in all urine samples (Table 5). These two proteins are expected to be abundant in urine; however, it was observed that their sequenced peptides were not always in the top 5 protein identifications per sample. Table 4 ranks the identified proteins that were most discriminatory between samples from tumor and non-tumor bearing patients by comparing the occurrence of a specific protein in urine samples obtained from tumor-bearing and non-tumor bearing individuals. Urinary serotransferrin and haptoglobin were associated with the presence of bladder cancer, but most discriminatory was alpha-1B-glycoprotein (A1BG-human). This protein was detected in all Con A-captured fractions of bladder cancer patients' samples, but more importantly, was never found to occur in non-tumor bearing patients' urine.

TABLE 3
Glycopeptides that were successfully identified in deglycosylated
fractions but went undetected in tryptic digests.

| | | Peptide | | | No. of |
|---|---|---|---|---|---|
| | Potential glycosylated | mass | Peptide | | samples |
| Protein Name | sites | MH+ | Seq | Glycosylation sites* | detected |
| Uromodulin | K.QDFN#ITDISLLEHR.L | 1701.85 | 319-332 | 322 N-linked (GlcNAc . . . ) | 2 |
| Prostaglandin-H2 | R.WFSAGLASN#SSWLR.E | 1582.77 | 45-56 | 51 N-linked (GlcNAc . . . ) | 5 |
| D-isomerase | K.SVVAPATDGGLN#LTSTFLR. | 1920.01 | 67-85 | 78 N-linked (GlcNAc . . . ) | 10 |
| | K | | | | |
| Lysosomal alpha- | K.LEN#LSSSEMGYTATLTR.T | 1873.90 | 138-154 | 140 N-linked (GlcNAc . . . ) | 5 |
| glucosidase | R.GVFITN#ETGQPLIGK.V | 1574.85 | 465-479 | 470 N-linked (GlcNAc . . . ) | 5 |
| | R.N#NTIVNELVR.V | 1172.63 | 882-890 | 882 N-linked (GlcNAc . . . ) | 7 |
| CD44 antigen | K.AFN#STLPTMAQMEK.A | 1569.74 | 55-68 | 57 N-linked (GlcNAc . . . ) | 8 |
| Kininogen-1 | K.YNSQN#QSNNQFVLYR.I | 1875.87 | 44-58 | 48 N-linked (GlcNAc . . . ) | 2 |
| | K.LNAENN#ATFYFK.I | 1432.68 | 289-300 | 294 N-linked (GlcNAc . . . ) | 7 |
| Alpha-1-antitrypsin | K.YLGN#ATAIFFLPDEGK.L | 1756.89 | 268-283 | 271 N-linked (GlcNAc . . . ) | 4 |
| Clusterin | R.LAN#LTQGEDQYYLR.V | 1725.94 | 372-385 | 374 N-linked (GlcNAc . . . ) | 4 |
| Polymeric-immunoglobulin | K.VPGN#VTAVLGETLK.V | 1398.79 | 466-479 | 469 N-linked (GlcNAc . . . ) | 6 |
| receptor | | | | | |
| Galectin-3-binding | R.ALGFEN#ATQALGR.A | 1348.69 | 64-76 | 69 N-linked (GlcNAc . . . ) | 3 |
| protein | | | | | |
| Cathepsin D | K.GSLSYLN#VTR.K | 1110.59 | 257-266 | 263 N-linked (GlcNAc . . . ) | 2 |
| Lysosomal acid | R.YEQLQN#ETR.Q | 1181.55 | 162-170 | 167 N-linked (GlcNAc . . . ) | 3 |
| phosphatase | | | | | |
| Ig alpha-2 chain C | K.TPLTAN#ITK.S | 959.55 | 200-208 | 205 N-linked (GlcNAc . . . ) | 3 |
| region | | | | | |
| Beta-glucuronidase | K.VVAN#GTGTQGQLK.V | 1273.69 | 269-281 | 272 N-linked (GlcNAc . . . ) | 4 |
| Haptoglobin | K.VVLHPN#YSQVDIGLIK.L | 1796.01 | 236-251 | 241 N-linked (GlcNAc . . . ) | 4 |
| Alpha-l-antichymotrypsin | K.YTGN#ASALFILPDQDK.M | 1753.88 | 268-283 | 271 N-linked (GlcNAc . . . ) | 2 |
| Lysosome-associated | K.IAVQFGPGFSWIAN#FTK.A | 1883.98 | 88-104 | 101 N-linked (GlcNAc . . . ) | 3 |
| membrane glycoprotein 2 | | | | | |
| Fibrillin-1 | K.TAIFAFN#ISHVSNK.V | 1549.81 | 2761-2774 | 2767 N-linked (GlcNAc . . . ) | 2 |
| Lysosome-associated | K.N#MTFDLPSDATVVLN#R.S | 1794.87 | 61-76 | 61,75 N-linked (GlcNAc . . . ) | 3 |
| membrane glycoprotein 1 | | | | | |
| Proactivator polypeptide | R.TN#STFVQALVEHVK.E | 1573.83 | 214-227 | 215 N-linked (GlcNAc . . . ) | 2 |

*Glycosylated sites as annotated in Swiss-Prot database

TABLE 4
Urinary proteins detected in >4 out of 5 samples tested in either a bladder tumor-bearing donor group (n = 5) or a non-tumor bearing
donor group (n = 5). The proteins were ranked based on their relative abundances in each group.

| | | Subcellular | | | Bladder | |
|---|---|---|---|---|---|---|
| Swiss-Prot ID | Protein name | location | Glycoproteins | Protein class | cancer (n = 5) | Normal (n = 5) |
| KLK1_HUMAN | Kallikrein-1 | secreted protein | o-, n-linked | Protease | 1 | 4 |
| ATRN_HUMAN | Attractin | secreted protein | n-linked | Immune system | 1 | 4 |
| ARSA_HUMAN | Arylsulfatase A | lysosome | n-linked | Enzyme | 3 | 4 |
| LAMP2_HUMAN | LAMP-2 | membrane | o-, n-linked | Adhesion | 3 | 4 |
| LYAG_HUMAN | Lysosomal alpha-glucosidase | lysosome | n-linked | Glycosidase | 3 | 4 |
| UROM_HUMAN | Uromodulin | secreted protein | n-linked | Unknown | 3 | 4 |
| ITIH4_HUMAN | Inter-alpha-inhibitor heavy chain 4 | extracellular | o-, n-linked | Protease inhibitor | 3 | 4 |
| CD44_HUMAN | CD44 antigen | membrane | n-linked | Adhesion | 4 | 4 |
| KNG1_HUMAN | Kininogen-1 | secreted protein | o-, n-linked | Protease inhibitor | 4 | 4 |
| KV2A_HUMAN | Ig kappa chain V-II region Cum | extracellular | no sites | Immune system | 4 | 4 |
| PIGR_HUMAN | Polymeric-immunoglobulin | secreted protein | n-linked | Carrier/transport protein | 4 | 4 |
| | receptor | | | | | |
| CD59_HUMAN | CD59 glycoprotein | membrane | o-, n-linked | Inhibitor | 4 | 5 |
| KAC_HUMAN | Ig kappa chain C region | extracellular | no sites | Immune system | 4 | 5 |
| LAC_HUMAN | Ig lambda chain C regions | extracellular | no sites | Immune system | 4 | 5 |
| PTGDS_HUMAN | Prostaglandin-H2 D-isomerase | secreted protein | n-linked | Enzyme | 4 | 5 |
| ZA2G_HUMAN | Zinc-alpha-2-glycoprotein | secreted protein | n-linked | Unknown | 4 | 5 |
| ALBU_HUMAN | Serum albumin | secreted protein | n-linked | Carrier/transport protein | 4 | 5 |
| IGHA1_HUMAN | Ig alpha-1 chain C region | secreted protein | o-, n-linked | Immune system | 5 | 4 |
| AMBP_HUMAN | Alpha-1-microglobulin | secreted protein | o-, n-linked | Protease inhibitor | 5 | 5 |
| KV3A_HUMAN | Ig kappa chain V-III region B6 | extracellular | no sites | Immune system | 4 | 3 |
| KV3B_HUMAN | Ig kappa chain V-III region SIE | extracellular | no sites | Immune system | 4 | 3 |
| CADH1_HUMAN | Epithelial-cadherin | membrane | n-linked | Adhesion | 4 | 3 |
| APOA_HUMAN | Apolipoprotein(a) | extracellular | o-, n-linked | Protease | 4 | 3 |
| A1AT_HUMAN | Alpha-1-antitrypsin | secreted protein | n-linked | Protease inhibitor | 4 | 2 |
| TRFE_HUMAN | Serotransferrin | secreted protein | o-, n-linked | Carrier/transport protein | 4 | 2 |
| HPT_HUMAN | Haptoglobin | secreted protein | n-linked | Carrier/transport protein | 5 | 1 |
| A1BG_HUMAN | Alpha-1B-glycoprotein | secreted protein | n-linked | Unknown | 5 | not detected |

TABLE 5
Urine specimen summary.

| Swiss-Prot ID | Protein Name | Subcellular location | Glycoprotein | T3 | T4 | T9 | T8 | T13 | N3 | N10 | N2 | N5 | N7 |
|---|---|---|---|---|---|---|---|---|---|---|---|---|---|
| A1AG1_HUMAN | Alpha-1-acid glycoprotein 1 | secreted protein | n-linked | | | 1 | 1 | 1 | | 1 | 1 | | |
| A1AG2_HUMAN | Alpha-1-acid glycoprotein 2 | secreted protein | n-linked | | | | | 1 | | | 1 | | |
| A1AT_HUMAN | Alpha-1-antitrypsin | secreted protein | n-linked | 1 | 1 | 1 | | 1 | | | 1 | | 1 |
| A1BG_HUMAN | Alpha-1B-glycoprotein | secreted protein | n-linked | 1 | 1 | 1 | 1 | 1 | | | | | |
| A2AP_HUMAN | Alpha-2-antiplasmin | secreted protein | n-linked | | | 1 | | | | | | | |
| A2GL_HUMAN | Leucine-rich alpha-2-glycoprotein | secreted protein | o-, n-linked | | | | | 1 | | | 1 | | |
| A2MG_HUMAN | Alpha-2-macroglobulin | extracellular | n-linked | 1 | | 1 | | | | | 1 | | |
| AACT_HUMAN | Alpha-1-antichymotrypsin | secreted protein | n-linked | 1 | 1 | 1 | | | | | 1 | | |
| ACTA_HUMAN | Actin, aortic smooth muscle | cytoplasm | potential | | | | | 1 | | | 1 | | |
| AFAM_HUMAN | Afamin | secreted protein | n-linked | 1 | | 1 | | | | | 1 | | |
| ALBU_HUMAN | Serum albumin | secreted protein | n-linked | 1 | | 1 | 1 | 1 | 1 | 1 | 1 | 1 | 1 |
| AMBP_HUMAN | AMBP protein | secreted protein | o-, n-linked | 1 | 1 | 1 | 1 | 1 | 1 | 1 | 1 | 1 | 1 |
| AMPN_HUMAN | Aminopeptidase N | membrane | n-linked | | | | | | | | | | |
| ANAG_HUMAN | Alpha-N-acetylglucosaminidase | lysosome | n-linked | | 1 | | | | 1 | | | | |
| ANT3_HUMAN | Antithrombin-III | secreted protein | n-linked | | | | | | | | | | |
| ANXA1_HUMAN | Annexin A1 | cytoplasm | potential | | 1 | | | | | | | | |
| AMYC_HUMAN | Alpha-amylase 2B | secreted protein | predicted | | | | | | | | | 1 | 1 |
| AMYP_HUMAN | Pancreatic alpha-amylase | secreted protein | n-linked | | | | | | | | | | |
| APOA_HUMAN | Apolipoprotein(a) | extracellular | o-, n-linked | 1 | 1 | | 1 | 1 | | 1 | 1 | 1 | |
| APOA1_HUMAN | Apolipoprotein A-I | secreted protein | no sites | | | 1 | | | | | 1 | | |
| APOA2_HUMAN | Apolipoprotein A-II | secreted protein | no sites | | | 1 | | | | | 1 | | |
| APOA4_HUMAN | Apolipoprotein A-IV | secreted protein | no sites | | | 1 | | | | | | | |
| APOC3_HUMAN | Apolipoprotein C-III | secreted protein | o-linked | | | 1 | | | | | | | |
| APOD_HUMAN | Apolipoprotein D | secreted protein | n-linked | | | | 1 | 1 | | 1 | | 1 | 1 |
| APOH_HUMAN | Beta-2-glycoprotein 1 | secreted protein | o-, n-linked | 1 | | 1 | | | 1 | 1 | 1 | | |
| ARSA_HUMAN | Arylsulfatase A | lysosome | n-linked | 1 | 1 | | 1 | | 1 | 1 | | 1 | 1 |
| ARSB_HUMAN | Arylsulfatase B | lysosome | n-linked | | | | | | | | | | |
| ASAH1_HUMAN | Acid ceramidase | lysosome | n-linked | | | | | | | | | | |
| ATM_HUMAN | Serine-protein kinase ATM | nucleus | potential | | | | | | | 1 | | | 1 |
| ATRN_HUMAN | Attractin | secreted protein | n-linked | | 1 | | | | 1 | 1 | | 1 | 1 |
| B2MG_HUMAN | Beta-2-microglobulin | secreted protein | no sites | | | | | 1 | | | 1 | | 1 |
| BASP_HUMAN | BASP1 protein | membrane | no sites | | | | | | | | | | |
| BGAL_HUMAN | Beta-galactosidase | lysosome | n-linked | | 1 | | 1 | | 1 | 1 | | | 1 |
| BGLR_HUMAN | Beta-glucuronidase | lysosome | n-linked | | | | 1 | | 1 | 1 | | | 1 |
| C4BP_HUMAN | C4b-binding protein alpha chain | extracellular | n-linked | | 1 | 1 | | | | | | | |
| CAD11_HUMAN | Cadherin-11 | membrane | n-linked | | | | 1 | | 1 | 1 | | | |
| CAD13_HUMAN | Cadherin-13 | membrane | n-linked | | 1 | | 1 | 1 | | | | 1 | |
| CADH1_HUMAN | Epithelial-cadherin | membrane | n-linked | 1 | 1 | | 1 | 1 | 1 | 1 | 1 | | |
| CADH2_HUMAN | Cadherin-2 | membrane | n-linked | 1 | 1 | | 1 | | 1 | 1 | | 1 | |
| CATC_HUMAN | Cathepsin C | lysosome | n-linked | | | | | | | | | | |
| CATD_HUMAN | Cathepsin D | lysosome | n-linked | 1 | 1 | | 1 | | | 1 | | | 1 |
| CATL_HUMAN | Cathepsin L | lysosome | n-linked | | | | | | | | | | |
| CATZ_HUMAN | Cathepsin Z | lysosome | n-linked | | 1 | | 1 | | | 1 | | | |
| CD14_HUMAN | Monocyte differentiation antigen | membrane | n-linked | | | | 1 | | | 1 | | | |
| | CD14 | | | | | | | | | | | | |
| CD44_HUMAN | CD44 antigen | membrane | n-linked | 1 | 1 | | 1 | 1 | 1 | 1 | | 1 | 1 |
| CD59_HUMAN | CD59 glycoprotein | membrane | o-, n-linked | 1 | 1 | | 1 | 1 | 1 | 1 | 1 | 1 | 1 |
| CERU_HUMAN | Ceruloplasmin | secreted protein | n-linked | 1 | 1 | 1 | | | | | | | |
| CFAH_HUMAN | Complement factor H | secreted protein | n-linked | | 1 | 1 | | | | | | | |
| CHD6_HUMAN | CHD-6 | nucleus | potential | | | | | | | | | | |
| CING_HUMAN | Cingulin | cytoplasm | potential | | 1 | 1 | | | | | | | |
| CLUS_HUMAN | Clusterin | secreted protein | n-linked | 1 | | 1 | 1 | 1 | | | | 1 | 1 |
| CO3_HUMAN | Complement C3 | secreted protein | n-linked | 1 | | 1 | | | | | 1 | | |
| CO4_HUMAN | Complement C4-B | secreted protein | n-linked | | 1 | 1 | | | | | | | |
| CO6A1_HUMAN | Collagen alpha-1(VI) chain | extracellular | n-linked | 1 | | | | | | | | | 1 |
| COCH_HUMAN | Cochlin | secreted protein | n-linked | | | | | | | | | | |
| COFA1_HUMAN | Collagen alpha-1(XV) chain | extracellular | n-linked | | | | | | | | | | |
| CYTC_HUMAN | Cystatin C | unknown | no sites | | | | | | | | | | |
| DERM_HUMAN | Dermatopontin | secreted protein | no sites | | | 1 | | | | | 1 | | |
| DDEF2_HUMAN | PAP | cytoplasm | potential | | | | | | | | | | |
| DIAC_HUMAN | Di-N-acetylchitobiase | lysosome | n-linked | | | | | | | | | | |
| DNAS1_HUMAN | Deoxyribonuclease-1 | secreted protein | n-linked | | | | | | | 1 | | | 1 |
| DPP4_HUMAN | Dipeptidyl peptidase 4 | secreted protein | n-linked | | | | | | | | | | |
| DSC2_HUMAN | Desmocollin-2 | membrane | n-linked | | 1 | | | | | | | | |
| DYN2_HUMAN | Dynamin-2 | cytoplasm | potential | | 1 | | | | 1 | | | | |
| EGF_HUMAN | Pro-epidermal growth factor | membrane | n-linked | 1 | 1 | | | | | | | 1 | 1 |
| ELNE_HUMAN | Leukocyte elastase | extracellular | n-linked | | | | | | | | | | |
| FBN1_HUMAN | Fibrillin-1 | extracellular | n-linked | | | | 1 | 1 | | | | 1 | 1 |
| FCG3A_HUMAN | IgG Fc receptor III-2 | secreted protein | n-linked | | 1 | | | | | | | | |
| FCG3B_HUMAN | IgG Fc receptor III-1 | secreted protein | n-linked | | 1 | | | | | | | | |
| FETUA_HUMAN | Alpha-2-HS-glycoprotein | secreted protein | o-, n-linked | | 1 | 1 | 1 | | | | | | |
| FIBA_HUMAN | Fibrinogen alpha chain | secreted protein | n-linked | 1 | | 1 | | | | | 1 | | |
| FIBB_HUMAN | Fibrinogen beta chain | secreted protein | n-linked | 1 | 1 | 1 | | | | | 1 | | |
| FIBG_HUMAN | Fibrinogen gamma chain | secreted protein | n-linked | 1 | | 1 | | | | | 1 | | |
| FINC_HUMAN | Fibronectin | secreted protein | o-, n-linked | | | 1 | 1 | | 1 | | | | |
| GDIS_HUMAN | Rho GDP-dissociation inhibitor 2 | cytoplasm | potential | | | | | | | | | | |
| GELS_HUMAN | Gelsolin | secreted protein | no sites | | | | 1 | 1 | | 1 | 1 | | 1 |
| GGH_HUMAN | Gamma-glutamyl hydrolase | secreted protein | n-linked | | | 1 | | 1 | 1 | | | | 1 |
| GILT_HUMAN | Gamma-interferon-inducible | lysosome | n-linked | | | | | | | | | | |
| | protein IP-30 | | | | | | | | | | | | |
| GL6S_HUMAN | N-acetylglucosamine-6-sulfatase | lysosome | n-linked | | 1 | | 1 | | 1 | 1 | | | |
| GP2_HUMAN | ZAP75 | secreted protein | n-linked | | 1 | | | | | | | | |
| GP73_HUMAN | Golgi phosphoprotein 2 | membrane | n-linked | | | | | | | | | | |
| HBA_HUMAN | Hemoglobin subunit alpha | — | no sites | | 1 | 1 | | | | | 1 | | |
| HBB_HUMAN | Hemoglobin subunit beta | — | n-linked | 1 | 1 | 1 | | | | | 1 | | |
| HBD_HUMAN | Hemoglobin subunit delta | — | no sites | | 1 | 1 | | | | | | | |
| HEMO_HUMAN | Hemopexin | secreted protein | o-, n-linked | 1 | | 1 | | | 1 | 1 | 1 | | |
| HEXA_HUMAN | Beta-hexosaminidase alpha chain | lysosome | n-linked | | | | | | | | | | |
| HEXB_HUMAN | Beta-hexosaminidase beta chain | lysosome | n-linked | | | | 1 | | 1 | | | | |
| HPT_HUMAN | Haptoglobin | secreted protein | n-linked | 1 | 1 | 1 | | 1 | | | 1 | | |
| HPTR_HUMAN | Haptoglobin-related protein | secreted protein | predicted | 1 | 1 | 1 | | | | 1 | 1 | | |
| HRG_HUMAN | Histidine-rich glycoprotein | secreted protein | n-linked | | 1 | 1 | | | | | | | |
| HV1A_HUMAN | Ig heavy chain V-I region EU | extracellular | no sites | 1 | | 1 | | | | | | | |
| HV3D_HUMAN | Ig heavy chain V-III region TIL | extracellular | no sites | | | 1 | | | | | | | |
| HV3E_HUMAN | Ig heavy chain V-III region BRO | extracellular | potential | | | 1 | | | | | 1 | | |
| HXA4_HUMAN | Homeobox protein Hox-A4 | nucleus | potential | | 1 | | | | | | | | |
| IBP7_HUMAN | IGFBP-7 | secreted protein | n-linked | 1 | | | | | | | | | |
| IC1_HUMAN | Plasma protease C1 inhibitor | secreted protein | o-, n-linked | 1 | | 1 | | | | | | | 1 |
| ICOSL_HUMAN | ICOS ligand | membrane | n-linked | | 1 | | | | | | | | |
| IGHA1_HUMAN | Ig alpha-1 chain C region | secreted protein | o-, n-linked | 1 | 1 | 1 | 1 | 1 | 1 | 1 | 1 | | 1 |
| IGHA2_HUMAN | Ig alpha-2 chain C region | secreted protein | n-linked | | | 1 | | | | | 1 | | |
| IGHG1_HUMAN | Ig gamma-1 chain C region | membrane | n-linked | 1 | 1 | 1 | | | | 1 | 1 | | |
| IGHG2_HUMAN | Ig gamma-2 chain C region | membrane | potential | | | 1 | | | | | 1 | | |
| IGJ_HUMAN | Immunoglobulin J chain | extracellular | n-linked | | | | | 1 | | 1 | | | |
| IPSP_HUMAN | Plasma serine protease inhibitor | secreted protein | n-linked | | | | | | | 1 | | 1 | 1 |
| ITIH1_HUMAN | Inter-alpha-inhibitor heavy chain 1 | extracellular | o-, n-linked | | 1 | 1 | | | | | | | |
| ITIH2_HUMAN | Inter-alpha-inhibitor heavy chain 2 | extracellular | o-, n-linked | | | 1 | | | | | | | |
| ITIH4_HUMAN | Inter-alpha-inhibitor heavy chain 4 | extracellular | o-, n-linked | | | 1 | 1 | 1 | | 1 | 1 | 1 | 1 |
| K0830_HUMAN | Zinc finger CCCH domain- | unknown | potential | | | 1 | 1 | | | | | | |
| | containing protein 13 | | | | | | | | | | | | |
| KAC_HUMAN | Ig kappa chain C region | extracellular | no sites | 1 | | 1 | 1 | 1 | 1 | 1 | 1 | 1 | 1 |
| KLK1_HUMAN | Kallikrein-1 | secreted protein | o-, n-linked | | | | | 1 | 1 | 1 | | 1 | 1 |
| KPYM_HUMAN | Pyruvate kinase isozymes M1/M2 | cytoplasm | no sites | | | | | | | | | | |
| KNG1_HUMAN | Kininogen-1 [Precursor] | secreted protein | o-, n-linked | 1 | 1 | 1 | 1 | | 1 | 1 | | 1 | 1 |
| KV1A_HUMAN | Ig kappa chain V-I region AG | extracellular | no sites | 1 | | | 1 | 1 | | | | | 1 |
| KV1N_HUMAN | Ig kappa chain V-I region OU | extracellular | no sites | 1 | 1 | | | 1 | | | | | |
| KV2A_HUMAN | Ig kappa chain V-II region Cum | extracellular | no sites | 1 | | 1 | 1 | 1 | 1 | 1 | 1 | | 1 |
| KV2C_HUMAN | Ig kappa chain V-II region MIL | extracellular | no sites | | | | | | | | | | |
| KV3A_HUMAN | Ig kappa chain V-III region B6 | extracellular | no sites | 1 | | 1 | 1 | 1 | 1 | | 1 | | 1 |
| KV3B_HUMAN | Ig kappa chain V-III region SIE | extracellular | no sites | 1 | 1 | | 1 | 1 | | 1 | 1 | | 1 |
| KV3C_HUMAN | Ig kappa chain V-III region NG9 | extracellular | no sites | | | | | | | | | | |
| LAC_HUMAN | Ig lambda chain C regions | extracellular | no sites | 1 | | 1 | 1 | 1 | 1 | 1 | 1 | 1 | 1 |
| LAMP1_HUMAN | LAMP-1 | membrane | o-, n-linked | | | | 1 | 1 | | 1 | | 1 | 1 |
| LAMP2_HUMAN | LAMP-2 | membrane | o-, n-linked | 1 | 1 | | 1 | | 1 | 1 | | 1 | 1 |
| LG3BP_HUMAN | Mac-2-binding protein | secreted protein | n-linked | 1 | 1 | | | 1 | | | | | 1 |
| LIFR_HUMAN | Leukemia inhibitory factor receptor | secreted protein | n-linked | | | 1 | | | | | | | |
| LMAN2_HUMAN | Vesicular integral-membrane | membrane | n-linked | | 1 | | | | | | | | |
| | protein VIP36 | | | | | | | | | | | | |
| LRP2_HUMAN | Glycoprotein 330 | membrane | n-linked | 1 | 1 | | | | | 1 | | | |
| LSAMP_HUMAN | Limbic system-associated | membrane | n-linked | | | | | | | | | | |
| | membrane protein | | | | | | | | | | | | |
| LU_HUMAN | Lutheran blood group glycoprotein | membrane | n-linked | | | | | | | | | | |
| LUM_HUMAN | Lumican | secreted protein | n-linked | | 1 | | | | | | | | |
| LV3A_HUMAN | Ig lambda chain V-III region SH | extracellular | no sites | | 1 | 1 | | | | | | | |
| LYAG_HUMAN | Lysosomal alpha-glucosidase | lysosome | n-linked | 1 | | | 1 | 1 | 1 | 1 | | 1 | 1 |
| MA2B2_HUMAN | Epididymis-specific alpha- | secreted protein | n-linked | | | | | | | | | | |
| | mannosidase | | | | | | | | | | | | |
| MASP2_HUMAN | MASP-2 | secreted protein | no sites | | | | | | | | | | |
| MGA_HUMAN | Maltase-glucoamylase, intestinal | membrane | n-linked | | 1 | | 1 | | 1 | | | | |
| MMP9_HUMAN | Matrix metalloproteinase-9 | extracellular | n-linked | | | | | | | | | | |
| MUC18_HUMAN | Cell surface glycoprotein MUC18 | membrane | n-linked | | 1 | | | | | | | | |
| MUCDL_HUMAN | Mucin and cadherin-like protein | membrane | o-, n-linked | | 1 | | | | | | | | |
| NAGAB_HUMAN | Alpha-galactosidase B | lysosome | n-linked | | | | 1 | | | | | | 1 |
| NARG1_HUMAN | NMDA receptor-regulated protein 1 | cytoplasm | potential | | | | | | | | | | |
| NCOR1_HUMAN | Nuclear receptor corepressor 1 | nucleus | potential | | | | 1 | 1 | | | | | |
| NEGR1_HUMAN | Neuronal growth regulator 1 | membrane | n-linked | | 1 | | | | | | | 1 | |
| NGAL_HUMAN | Neutrophil gelatinase-associated | secreted protein | n-linked | | | | | | | | | | |
| | lipocalin | | | | | | | | | | | | |
| NIDO_HUMAN | Nidogen-1 | secreted protein | n-linked | | | | | | | 1 | | | 1 |
| NTRI_HUMAN | Neurotrimin | membrane | n-linked | | | | | | 1 | | | | |
| ORN_HUMAN | Oligoribonuclease, mitochondrial | nucleus | no sites | | | | | | | | | | |
| OSTP_HUMAN | Osteopontin | secreted protein | o-, n-linked | 1 | 1 | | 1 | | | 1 | | | 1 |
| PCDH1_HUMAN | Protocadherin-1 | membrane | n-linked | | 1 | | | | | | | | |
| PCP_HUMAN | Lysosomal Pro-X | lysosome | n-linked | | | | | | | | | | |
| | carboxypeptidase | | | | | | | | | | | | |
| PEPA_HUMAN | Pepsin A | secreted protein | no sites | | | | | | | | | | |
| PERM_HUMAN | Myeloperoxidase | lysosome | n-linked | | | | | | | | | | |
| PGBM_HUMAN | HSPG | secreted protein | o-, n-linked | | 1 | | 1 | 1 | | 1 | | 1 | 1 |
| PIGR_HUMAN | Polymeric-immunoglobulin | secreted protein | n-linked | 1 | 1 | | 1 | 1 | 1 | 1 | | 1 | 1 |
| | receptor | | | | | | | | | | | | |
| PPAL_HUMAN | Lysosomal acid phosphatase | lysosome | n-linked | | | | 1 | | 1 | 1 | | | 1 |
| PPGB_HUMAN | Lysosomal protective protein | lysosome | n-linked | | 1 | | 1 | | 1 | 1 | | | 1 |
| PRSS8_HUMAN | Prostasin | secreted protein | n-linked | | | | | | | | | | 1 |
| PTGDS_HUMAN | Prostaglandin-H2 D-isomerase | secreted protein | n-linked | 1 | 1 | | 1 | 1 | 1 | 1 | 1 | 1 | 1 |
| QPCT_HUMAN | Glutaminyl-peptide | unknown | n-linked | | | | 1 | | | 1 | | 1 | 1 |
| | cyclotransferase | | | | | | | | | | | | |
| RS15_HUMAN | 40S ribosomal protein S15 | cytoplasm | no sites | | | | | | | | | | |
| S100P_HUMAN | Protein S100-P | cytoplasm | no sites | | | | | 1 | | 1 | | | |
| S10A6_HUMAN | Protein S100-A6 | cytoplasm | no sites | | | | | | | | | | |
| SAMP_HUMAN | Serum amyloid P-component | secreted protein | n-linked | | | 1 | | | | | | | |
| SAP_HUMAN | Proactivator polypeptide | lysosome | n-linked | | | | 1 | | | 1 | | 1 | 1 |
| SAP3_HUMAN | Ganglioside GM2 activator | lysosome | n-linked | | | | | | | | | | |
| SCG1_HUMAN | Secretogranin-1 | secretory granules | predicted | | | | | | | | | 1 | 1 |
| SDC1_HUMAN | Syndecan-1 | membrane | o-, n-linked | 1 | | | 1 | | | | | | |
| SDC4_HUMAN | Syndecan-4 | membrane | o-linked | 1 | 1 | | 1 | | 1 | 1 | | 1 | |
| SH3L3_HUMAN | SH3 domain-binding protein 1 | cytoplasm | no sites | | | | 1 | 1 | | 1 | | | 1 |
| SHPS1_HUMAN | Signal-regulatory protein alpha-1 | membrane | n-linked | 1 | 1 | | 1 | | 1 | 1 | | 1 | |
| SIRB1_HUMAN | Signal-regulatory protein beta-1 | membrane | n-linked | | 1 | | | | 1 | | | | |
| SODE_HUMAN | Extracellular superoxide dismutase | secreted protein | n-linked | | 1 | | | | | | | | |
| | [Cu—Zn] | | | | | | | | | | | | |
| SULF2_HUMAN | Extracellular sulfatase Sulf-2 | extracellular | n-linked | | | | | | | | | | |
| THIO_HUMAN | Thioredoxin | cytoplasm | no sites | | | | | | | | | | |
| TETN_HUMAN | Tetranectin | secreted protein | o-linked | | | | | | | | | | |
| TPP1_HUMAN | Tripeptidyl-peptidase 1 | lysosome | predicted | | | | 1 | | | 1 | | | 1 |
| TRFE_HUMAN | Serotransferrin | secreted protein | o-, n-linked | 1 | 1 | 1 | | 1 | | 1 | 1 | | |
| TRFL_HUMAN | Lactotransferrin | secreted protein | n-linked | | | | | | | | | | |
| TTLL7_HUMAN | Tubulin--tyrosine ligase-like | unknown | potential | | | 1 | | | | | 1 | | |
| | protein 7 | | | | | | | | | | | | |
| VMO1_HUMAN | Vitelline membrane outer layer | secreted protein | no sites | | | | 1 | | | | | 1 | 1 |
| | protein 1 homolog | | | | | | | | | | | | |
| UBIQ_HUMAN | Ubiquitin | cytoplasm | no sites | | | | | | | | | | |
| UD12_HUMAN | UDP-glucuronosyltransferase 1-2 | microsome | n-linked | | | | 1 | 1 | | | | | |
| UFO_HUMAN | Tyrosine-protein kinase receptor | membrane | n-linked | 1 | 1 | | | | 1 | 1 | | 1 | |
| | UFO | | | | | | | | | | | | |
| UROM_HUMAN | Uromodulin | secreted protein | n-linked | 1 | 1 | | 1 | | 1 | 1 | | 1 | 1 |
| UTER_HUMAN | Uteroglobin | secreted protein | no sites | | | | | | | | | | |
| VEZA_HUMAN | Vezatin | membrane | potential | 1 | | 1 | | | | | | | |
| VTNC_HUMAN | Vitronectin | secreted protein | n-linked | 1 | | 1 | | | | | | | |
| WTAP_HUMAN | WT1-associated protein | nucleus | potential | | | | | | | 1 | | 1 | |
| ZA2G_HUMAN | Zinc-alpha-2-glycoprotein | secreted protein | n-linked | 1 | 1 | | 1 | 1 | 1 | 1 | 1 | 1 | 1 |
| Total ID/sample | | | | 56 | 68 | 58 | 69 | 49 | 49 | 60 | 49 | 41 | 61 |

In the experiments described herein, the analysis of one specific fraction of the urinary proteome, that of glycosylated proteins, was the focus. Selection and concentration of one component of the sample proteome reduced the complexity of the sample, which in turn enabled the rapid and accurate identification of multiple unique proteins in sample volumes that are routinely, and non-invasively, obtained in the clinic. Protein glycosylation has long been recognized as one of the most prevalent posttranslational modifications (Parodi, A. J. Annu Rev Biochem, 2000, 69:69-93), playing a fundamental role in many biological processes such as immune response and cellular regulation (Rudd, P. M. et al. Science, 2001, 291(5512):2370-6). Accordingly, the alteration in protein glycosylation which occurs through varying the heterogeneity of glycosylation sites or changing glycan structure of proteins on the cell surface and in body fluids has been shown to correlate with the development of numerous disease states, including cancer (Block, T. M. et al. Proc Natl Acad Sci USA, 2005, 102(3):779-84). Indeed, many clinical biomarkers and therapeutic targets in cancer are glycoproteins (Dube, D. H. and Bertozzi, C. R. Nat Rev Drug Discov, 2005, 4(6):477-88) such as CAl25 in ovarian cancer, Her2/neu in breast cancer, and prostate-specific antigen (PSA) in prostate cancer. PSA is one of the best characterized examples of a secreted glycoprotein used in cancer diagnostics, and its glycoforms have been described (Prakash, S. and Robbins, P. W. Glycobiology, 2000, 10(2):173-6). Such carbohydrate differences allow a distinction to be made between proteins from normal and tumor origins and suggest a valuable biochemical tool for diagnosis (Peracaula, R. et al. Glycobiology, 2003, 13(6):457-70).

The use of Con A affinity chromatography combined with a powerful nano-LC/MS/MS platform provided an efficient capture of N-glycoproteins from a relatively complex sample, and enabled the identification of 186 unique proteins. This combinatorial approach provides high sensitivity and with relatively moderate labor demands should facilitate the identification of potential biomarkers of disease from body fluids. This comparative study of a panel of clinical samples revealed that differential urinary glycoprotein profiles exist that can distinguish bladder cancer-bearing patients from individuals with non-cancer conditions. It is important to note that only relatively high abundance peptides/proteins will be positively identified by LC/MS/MS analysis. In particular, ion suppression can occur when many peptides elute from the column simultaneously. Increasing the number of sequenced peptides per scan or using mass exclusion may increase the number of identified proteins, but these improvements are still restricted by the MS performance. Obtaining large sequence coverage as well as sequencing large peptides is also difficult with the shotgun approach. In this study, repeated analysis of sample enabled the present inventors to detect low level peptides/proteins and obtain confident ids of N-linked glycoproteins in a small amount of patient urine. In addition to high Xcorr criteria for peptide/protein assignment, restricted SEQUEST scores and manual analysis were also performed to assure accuracy of our assignments. Using this concerted approach, the present inventors were able to distinguish the marked differences between a group of healthy individuals and cancer-bearing patients and propose potential diagnostic biomarkers.

Of the 186 proteins identified in this study, 146 have been detected in urine using other techniques (Adachi, J. et al. Genome Biol, 2006, 7(9):R80; Zerefos, P. G. et al. Proteomics, 2006, 6(15):4346-55), thus, the present inventors have added a significant list to the urinary proteome database. Several potential novel glycosylated peptides were identified in this study, but these would require confirmational studies focused upon the detailed analysis of specific proteins. Even though all patients visiting the clinic presented with hematuria, the presence of several glycoproteins were associated with samples obtained from patients with transitional cell carcinoma of the bladder. The most discriminatory protein associated with bladder cancer in this pilot study was alpha-1B-glycoprotein (A1BG). Although the human A1BG protein was purified and sequenced in 1986, its physiological role is unknown. Ishioka et al. determined the complete amino acid sequence of A1BG, and showed it to consist of a single polypeptide chain N-linked to four glucosamine oligosaccharides. Analysis of the amino acid sequence revealed significant homology to variable regions of certain immunoglobulin light and heavy chains, and to other members of the immunoglobulin supergene family (Ishioka, N. et al, Proc Natl Acad Sci USA, 1986, 83(8):2363-7). No specific function has been ascribed to A1BG to date, but an indication of a function comes from the characterization of the opossum A1BG homolog, oprin (Catanese, J. J. and Kress, L. F. Biochemistry, 1992, 31(2):410-8). Oprin is a metalloproteinase inhibitor, which in some properties, but not in sequence, resembles tissue inhibitor of metalloproteinases (TIMPs), a family of proteins with complex roles in tumor progression and angiogenesis (Chirco, R. et al. Cancer Metastasis Rev, 2006, 25(1):99-113). An association of A1BG expression with cancer has been described in one previous study. In a study that utilized expressed sequence tag (EST) profiling of cDNA libraries, Yoon et al. identified A1BG as one of 14 genes confirmed to be highly expressed in human hepatocellular carcinoma (HCC) cell lines and liver tumor tissue specimens (Yoon, S. Y. et al. Int J Oncol, 2006, 29(2):315-27). The reported correlation of A1BG expression with liver cancer further supports the notion that this protein is a useful biomarker for bladder cancer.

Beyond the investigation of individual proteins identified here, the described technical approach will facilitate a more focused analysis of clinically relevant samples, such as urine and other bodily fluids, which in turn will lead to the identification of reliable biomarkers for improved detection, surveillance and screening regimens for a range of diseases.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for detecting or diagnosing a urogenital related cancer in a subject, comprising detecting the presence of and/or quantifying the level of at least one biomarker protein or a nucleic acid encoding a biomarker listed in Table 4 in a biological sample from the subject, wherein the presence of the biomarker, or a level or concentration of the biomarker above a pre-determined threshold is indicative of cancer in the subject.

2. The method according to claim 1, wherein the detecting comprises: (a) contacting the biological sample with a binding agent (or binding agents) that binds the biomarker protein (or biomarker proteins) to form a complex (or complexes); and (b) detecting and/or quantifying the complex(es); and correlating the detected complex(es) to the amount of biomarker protein(s) in the sample, wherein the presence of one or more biomarkers, or the presence of elevated levels of biomarker protein(s), is indicative of cancer.

3. The method according to claim 2, wherein the detecting of step (b) further comprises linking or incorporating a label onto the agent, or using ELISA-based immunoenzymatic detection.

4. The method according to claim 1, wherein said method further comprises comparing the level of one or more biomarkers in the biological sample with the level of biomarker present in a normal control sample, wherein a higher level of biomarker in the sample as compared to the level in the normal control sample is indicative of the presence of cancer.

5. The method according to claim 1, wherein the cancer is bladder cancer.

6. The method according to claim 1, wherein the biological sample is tissues or extracts, including cells (e.g., tumor cells) and physiological fluids, such as, for example, whole blood, plasma, serum, peritoneal fluid, ascites, and the like.

7. The method according to claim 1, wherein the biological sample is urine.

8. The method according to claim 1, wherein the subject is human.

9. The method according to claim 1, wherein the at least one biomarker is Alpha-1B-glycoprotein, Haptoglobin, Serotransferrin, or Alpha-1-antitrypsin, or any combination thereof.

10. The method according to claim 1, wherein the nucleic acid is detected using an oligonucleotide that is complementary with at least a portion of the nucleic acid.

11. The method according to claim 1, wherein the nucleic acid is detected using a nucleic acid hybridization method.

12. The method according to claim 11, wherein the nucleic acid hybridization method is Northern blot analysis, dot blotting, Southern blot analysis, fluorescence in situ hybridization (FISH), or polymerase chain reaction (PCR) analysis.

13. The method according to claim 12, wherein the PCR is reverse transcription PCR (RT-PCR).

14. The method according to claim 1, wherein the protein is detected using an aptamer, peptide, or antibody that binds to the protein.

15. The method according to claim 1, wherein the protein is detected using an electrophoretic, chromatographic, or spectrometric method.

16. The method according to claim 1, wherein the protein is detected using mass spectrometry.

17. A method for prognostic evaluation of a subject having, or suspected of having, a urogenital related cancer, comprising: a) determining the level of one or more cancer biomarkers listed in Table 4 in a biological sample obtained from the subject; b) comparing the level determined in step (a) to a level or range of the one or more cancer biomarkers known to be present in a biological sample obtained from a normal subject that does not have cancer; and c) determining the prognosis of the subject based on the comparison of step (b), wherein a high level of the one or more cancer biomarkers in step (a) indicates a more aggressive or more metastatic form of cancer.

18. The method according to claim 17, wherein the cancer is bladder cancer.

19. The method according to claim 17, wherein the biological sample is tissues or extracts, including cells (e.g., tumor cells) and physiological fluids, such as, for example, whole blood, plasma, serum, peritoneal fluid, ascites, and the like.

20. The method according to claim 17, wherein the biological sample is urine.

21. The method according to claim 17, wherein the subject is human.

22. The method according to claim 17, wherein the at least one biomarker is Alpha-1B-glycoprotein, Haptoglobin, Serotransferrin, or Alpha-1-antitrypsin, or any combination thereof.

23. A method for detecting a urogenital cancer comprising:
(a) incubating a biological sample with a first antibody specific for at least one cancer biomarker polypeptide listed in Table 4 which is directly or indirectly labeled with a detectable substance, and a second antibody specific for the biomarker polypeptide which is immobilized;
(b) separating the first antibody from the second antibody to provide a first antibody phase and a second antibody phase;
(c) detecting the detectable substance in the first or second antibody phase thereby quantitating the biomarker in the sample; and
(d) comparing the quantitated biomarker with a standard.

24. The method according to claim 23, wherein the cancer is bladder cancer.

25. The method according to claim 23, wherein the biological sample is tissues or extracts, including cells (e.g., tumor cells) and physiological fluids, such as, for example, whole blood, plasma, serum, peritoneal fluid, ascites, and the like.

26. The method according to claim 23, wherein the biological sample is urine.

27. The method according to claim 23, wherein the subject is human.

28. The method according to claim 23, wherein the at least one biomarker is Alpha-1B-glycoprotein, Haptoglobin, Serotransferrin, or Alpha-1-antitrypsin, or any combination thereof.

29. A kit for monitoring, detecting, or diagnosing a urogenital cancer, said kit comprising in one or more containers:
a) one or more agents for detecting or quantifying presence of one or more biomarker proteins or nucleic acids listed in Table 4.

30. The kit according to claim 29, wherein the at least one biomarker is Alpha-1B-glycoprotein, Haptoglobin, Serotransferrin, or Alpha-1-antitrypsin, or any combination thereof.

31. The kit according to claim 29, wherein said kit further comprises a container for collecting a biological sample from a subject.
